(19) United States
(12) Patent Application Publication
(10) Pub. No.: US 2026/0198526 A1
(43) Pub. Date: Jul. 16, 2026

(54) METHOD FOR PROMOTING DIFFERENTIATION OF ANIMAL CELL

(71) Applicant: Ajinomoto Co., Inc., Tokyo (JP)

(72) Inventors: Shimpei OGAWA, Kanagawa (JP); Seiji KITAJIMA, Kanagawa (JP)

(73) Assignee: Ajinomoto Co., Inc., Tokyo (JP)

(21) Appl. No.: 19/565,831

(22) Filed: Mar. 13, 2026

Related U.S. Application Data
(63) Continuation of application No. PCT/JP2024/032850, filed on Sep. 13, 2024.

(30) Foreign Application Priority Data
Sep. 15, 2023 (JP) 2023-149689

(51) Int. Cl. A23J 3/22; A23J 3/04; C12N 5/077

(52) U.S. Cl. CPC A23J 3/227; A23J 3/04; C12N 5/0659; C12N 2506/1323

(57) ABSTRACT

An object is to provide a method for promoting the differentiation of animal cells and related technology, which is achieved by culturing animal cells in the presence of a taste substance.

### CROSS-REFERENCE TO RELATED APPLICATIONS

This application is a Continuation of, and claims priority under 35 U.S.C. § 120 to, International Application No. PCT/JP2024/032850, filed Sep. 13, 2024, and claims priority therethrough under 35 U.S.C. § 119 to Japanese Patent Application No. 2023-149689, filed Sep. 15, 2023, the entireties of which are incorporated by reference herein.

### BACKGROUND OF THE INVENTION

### Technical Field

The present invention relates to a method for promoting the differentiation of animal cells and related technology.

### Background Art

For culturing animal cells, a medium containing serum, such as fetal bovine serum, is widely used. However, from the standpoints of safety, economy, and the like, the development of a serum-free medium that does not contain serum is desired. For example, serum-free media have been developed in which serum is replaced with alternative components such as growth factors.

Furthermore, it is known that intracellular calcium ions are involved in muscle differentiation (Non-Patent Literature 1).

It is also known that taste substances cause an increase in intracellular calcium ion concentration in taste cells through binding with taste receptors on the taste cells (Non-Patent Literature 2).

### CITATION LIST

### Non-Patent Literature

Non-Patent Literature 1: Sansrity Sinha et al., Ca2+ as a coordinator of skeletal muscle differentiation, fusion and contraction. FEBS J. 2022 November; 289 (21): 6531-6542.
Non-Patent Literature 2: Rais Ahmad and Julie E Dalziel, G Protein-Coupled Receptors in Taste Physiology and Pharmacology. Front Pharmacol. 2020 Nov. 30; 11:587664.

### SUMMARY OF INVENTION

An object of the present invention is to provide a method for promoting the differentiation of animal cells and related technology.

The present inventors have found that the differentiation of animal cells can be promoted in the absence of serum by using a taste substance, thereby completing the present invention.

That is, the present invention can be exemplified as follows.

[1]

A composition for promoting differentiation of animal cells, comprising a taste substance.

[2]

The composition according to [1], which is a medium or a medium supplement.

[3]

The composition according to [1] or [2], wherein the medium is a basal medium, a fed-batch medium, or a perfusion medium.

[4]

The composition according to any one of [1] to [3], wherein the taste substance is at least one component selected from the group consisting of a sweet substance, a salty substance, a sour substance, a bitter substance, an umami substance, a fatty taste substance, a kokumi substance, a succinate receptor agonist, and a GABA-B receptor agonist.

[5]

The composition according to [4], wherein
the sweet substance is at least one component selected from the group consisting of a sugar, a sugar alcohol, a high-intensity sweetener, and an amino acid,
the salty substance is at least one inorganic salt,
the sour substance is at least one component selected from the group consisting of an organic acid, an inorganic acid, and an amino acid,
the bitter substance is at least one component selected from the group consisting of an amino acid, a polyphenol, an alkaloid, a terpenoid, an inorganic salt, diphenidol, and salicin, the umami substance is at least one component selected from the group consisting of an amino acid, a nucleic acid, an organic acid, an amide compound, and a high-potency umami substance,
the fatty taste substance is at least one component selected from the group consisting of a fatty acid and a fatty acid receptor agonist,
the kokumi substance is at least one component selected from the group consisting of a γ-glutamyl peptide and a calcium-sensing receptor agonist,
the succinate receptor agonist is succinic acid, and
the GABA-B receptor agonist is GABA and/or SKF97541.
   [6]

The composition according to any one of [1] to [5], wherein the taste substance is at least one component selected from the group consisting of a sweet taste receptor agonist, a salty taste receptor agonist, a sour taste receptor agonist, a bitter taste receptor agonist, an umami taste receptor agonist, a fatty acid receptor agonist, a calcium-sensing receptor agonist, a succinate receptor agonist, and a GABA-B receptor agonist.

[7]

The composition according to [6], wherein
the sweet taste receptor is T1R2/3,
the salty taste receptor is ENaC, TMC4, or TMC6,
the sour taste receptor is GPR91, PKD2L1, PKD1L3, or Otop1,
the bitter taste receptor is T2R,
the umami taste receptor is T1R1/3,
the fatty acid receptor is GPR120, and
the succinate receptor is GPR91.
   [8]

The composition according to any one of [1] to [7], wherein the taste substance is at least one component selected from the group consisting of ATF100, sucralose, cinacalcet, TUG-891, diphenidol, salicin, SKF97541, and succinic acid.

[9]

The composition according to any one of [1] to [8], wherein the animal is a chicken and the taste substance is salicin.

[10]

The composition according to any one of [1] to [9], wherein the taste substance is a taste substance other than a sugar, an amino acid, and calcium.

[11]

The composition according to any one of [1] to [10], wherein the animal is a mammal, a bird, a fish, or a crustacean.

[12]

The composition according to any one of [1] to [11], wherein the animal is an animal for meat production.

[13]

The composition according to any one of [1] to [12], wherein the animal is a cow or a chicken.

[14]

The composition according to any one of [1] to [13], wherein the differentiation is muscle differentiation.

[15]

The composition according to any one of [1] to [14], wherein the cell is a satellite cell or a myoblast.

[16]

The composition according to any one of [1] to [15], which is substantially free of serum.

[17]

The composition according to any one of [1] to [16], which is substantially free of animal-derived albumin.

[18]

A method for producing cultured meat, comprising the step of:
culturing animal cells in the presence of a taste substance to differentiate the animal cells.
   [19]

A method for promoting differentiation of animal cells, comprising the step of:
culturing animal cells in the presence of a taste substance to differentiate the animal cells.
   [20]

The method according to or [19], wherein the taste substance is at least one component selected from the group consisting of a sweet substance, a salty substance, a sour substance, a bitter substance, an umami substance, a fatty taste substance, a kokumi substance, a succinate receptor agonist, and a GABA-B receptor agonist.

[21]

The method according to [20], wherein
the sweet substance is at least one component selected from the group consisting of a sugar, a sugar alcohol, a high-intensity sweetener, and an amino acid,
the salty substance is at least one inorganic salt,
the sour substance is at least one component selected from the group consisting of an organic acid, an inorganic acid, and an amino acid,
the bitter substance is at least one component selected from the group consisting of an amino acid, a polyphenol, an alkaloid, a terpenoid, an inorganic salt, diphenidol, and salicin, the umami substance is at least one component selected from the group consisting of an amino acid, a nucleic acid, an organic acid, an amide compound, and a high-potency umami substance,
the fatty taste substance is at least one component selected from the group consisting of a fatty acid and a fatty acid receptor agonist,
the kokumi substance is at least one component selected from the group consisting of a γ-glutamyl peptide and a calcium-sensing receptor agonist,
the succinate receptor agonist is succinic acid, and
the GABA-B receptor agonist is GABA and/or SKF97541.
   [22]

The method according to any one of to [21], wherein the taste substance is at least one component selected from the group consisting of a sweet taste receptor agonist, a salty taste receptor agonist, a sour taste receptor agonist, a bitter taste receptor agonist, an umami taste receptor agonist, a fatty acid receptor agonist, a calcium-sensing receptor agonist, a succinate receptor agonist, and a GABA-B receptor agonist.

[23]

The method according to [22], wherein
the sweet taste receptor is T1R2/3,
the salty taste receptor is ENaC, TMC4, or TMC6,
the sour taste receptor is GPR91, PKD2L1, PKD1L3, or Otop1,
the bitter taste receptor is T2R,
the umami taste receptor is T1R1/3,
the fatty acid receptor is GPR120, and
the succinate receptor is GPR91.
   [24]

The method according to any one of to [23], wherein the taste substance is at least one component selected from the group consisting of ATF100, sucralose, cinacalcet, TUG-891, diphenidol, salicin, SKF97541, and succinic acid.

[25]

The method according to any one of to [24], wherein the animal is a chicken and the taste substance is salicin.

[26]

The method according to any one of to [25], wherein the taste substance is a taste substance other than a sugar, an amino acid, and calcium.

[27]

The method according to any one of to [26], wherein the concentration of the taste substance in the medium during culturing is from 0.05 nM to 500 mM.

[28]

The method according to [20], satisfying one, two, three, four, five, six, seven, eight, or nine conditions selected from the following (1) to (9):
(1) the concentration of the sweet substance in the medium during culturing is from 100 nM to 2 mM;
(2) the concentration of the salty substance in the medium during culturing is from 0.05 nM to 500 mM;
(3) the concentration of the sour substance in the medium during culturing is from 10 nM to 100 μM;
(4) the concentration of the bitter substance in the medium during culturing is from 10 nM to 200 μM or 5 μM to 100 mM;
(5) the concentration of the umami substance in the medium during culturing is from 0.1 nM to 2 μM;
(6) the concentration of the fatty taste substance in the medium during culturing is from 10 nM to 100 μM;
(7) the concentration of the kokumi substance in the medium during culturing is from 0.1 nM to 2 μM;
(8) the concentration of the succinate receptor agonist in the medium during culturing is from 10 nM to 100 μM;
(9) the concentration of the GABA-B receptor agonist in the medium during culturing is from 10 nM to 100 μM.
   [29]

The method according to [22], satisfying one, two, three, four, five, six, seven, eight, or nine conditions selected from the following (1) to (9):
(1) the concentration of the sweet taste receptor agonist in the medium during culturing is from 100 nM to 2 mM;
(2) the concentration of the salty taste receptor agonist in the medium during culturing is from 0.05 nM to 500 mM;
(3) the concentration of the sour taste receptor agonist in the medium during culturing is from 10 nM to 100 μM;
(4) the concentration of the bitter taste receptor agonist in the medium during culturing is from 10 nM to 200 μM or 5 μM to 100 mM;
(5) the concentration of the umami taste receptor agonist in the medium during culturing is from 0.1 nM to 2 μM;
(6) the concentration of the fatty acid receptor agonist in the medium during culturing is from 10 nM to 100 μM;
(7) the concentration of the calcium-sensing receptor agonist in the medium during culturing is from 0.1 nM to 2 μM;
(8) the concentration of the succinate receptor agonist in the medium during culturing is from 10 nM to 100 μM;
(9) the concentration of the GABA-B receptor agonist in the medium during culturing is from 10 nM to 100 μM.
   [30]

The method according to any one of to [29], wherein the animal is a mammal, a bird, a fish, or a crustacean.

[31]

The method according to any one of to [30], wherein the animal is an animal for meat production.

[32]

The method according to any one of to [31], wherein the animal is a cow or a chicken.

[33]

The method according to any one of to [32], wherein the differentiation is muscle differentiation.

[34]

The method according to any one of to [33], wherein the cell is a satellite cell or a myoblast.

[35]

The method according to any one of to [34], wherein the culturing is performed in a medium substantially free of serum.

[36]

The method according to any one of to [35], wherein the culturing is performed in a medium substantially free of animal-derived albumin.

### BRIEF DESCRIPTION OF THE DRAWINGS

FIG. 1 shows the differentiation of bovine muscle stem cells in a serum-free medium in the presence of various taste substances.

FIG. 2 shows the differentiation of chicken muscle stem cells in a serum-free medium in the presence of various taste substances.

### DESCRIPTION OF EMBODIMENTS

Hereinafter, the present invention will be described in detail.

### <1> Active Ingredient

In the present invention, a taste substance is used as an active ingredient.

A taste substance may also be referred to as an “active ingredient”.

The active ingredient can be used for culturing animal cells. Specifically, animal cells can be cultured in the presence of the active ingredient. More specifically, animal cells can be cultured and differentiated in the presence of the active ingredient.

By using the active ingredient, specifically by culturing animal cells in the presence of the active ingredient, the differentiation of animal cells may be promoted, that is, an effect of promoting the differentiation of animal cells may be obtained. This effect is also referred to as a “differentiation-promoting effect”. In other words, the active ingredient may have a function of promoting the differentiation of animal cells. This function is also referred to as a “differentiation-promoting function”. That is, by culturing animal cells in the presence of the active ingredient, the differentiation of animal cells may be promoted as compared with the case where animal cells are cultured in the absence of the active ingredient. The case where animal cells are cultured in the absence of the active ingredient includes the case where animal cells are cultured under the same conditions as in the case where animal cells are cultured in the presence of the active ingredient, except that the active ingredient is not used. The differentiation-promoting effect may be obtained, for example, when animal cells are cultured under conditions where serum is substantially absent (that is, when animal cells are cultured in a medium substantially free of serum). The differentiation-promoting effect may be obtained, for example, when animal cells are cultured under conditions where animal-derived albumin is substantially absent (that is, when animal cells are cultured in a medium substantially free of animal-derived albumin). The differentiation-promoting effect may be obtained, for example, when animal cells are cultured under conditions where serum and animal-derived albumin are substantially absent (that is, when animal cells are cultured in a medium substantially free of serum and animal-derived albumin). Examples of the differentiation include muscle differentiation. Examples of the promotion of differentiation include an increase in the differentiation rate and an increase in the ratio of differentiated cells. The differentiation-promoting effect can be confirmed by measuring and comparing the differentiation of animal cells (for example, the differentiation rate and the ratio of differentiated cells) in the presence and absence of the active ingredient. The differentiation of animal cells can be measured, for example, using the expression of a protein that serves as an index of differentiation as an index. For example, in muscle differentiation, Sproutyl, Pax7, MyoD, Myogenin, and Myosin heavy chain (MHC) may be expressed in this order as differentiation progresses (Manuel Schmidt et al. Adult stem cells at work: regenerating skeletal muscle. Cell Mol Life Sci. 2019 July; 76 (13): 2559-2570.). In addition, the differentiation of animal cells can be measured, for example, in the case of muscle differentiation, using the Fusion Index as an index.

In one embodiment, a product may be produced by culturing animal cells. Examples of the product include cultured meat. When a product is produced by culturing animal cells, the production of the product may be improved by using the active ingredient. That is, by culturing animal cells in the presence of the active ingredient, the production of the product may be improved as compared with the case where animal cells are cultured in the absence of the active ingredient. For example, the production of the product may be improved by promoting the differentiation of animal cells by using the active ingredient. That is, one embodiment of the differentiation-promoting effect may be an effect of improving the production of the product. The effect of improving the production of the product may be obtained, for example, when animal cells are cultured under conditions where serum is substantially absent (that is, when animal cells are cultured in a medium substantially free of serum). The effect of improving the production of the product may be obtained, for example, when animal cells are cultured under conditions where animal-derived albumin is substantially absent (that is, when animal cells are cultured in a medium substantially free of animal-derived albumin). The effect of improving the production of the product may be obtained, for example, when animal cells are cultured under conditions where serum and animal-derived albumin are substantially absent (that is, when animal cells are cultured in a medium substantially free of serum and animal-derived albumin). Examples of the improvement in production include an improvement in the production amount and an improvement in the production rate. When a product is produced by culturing animal cells, a product to which a taste corresponding to the taste component is imparted or enhanced may be produced.

As the active ingredient, one type of taste substance may be used, or two or more types of taste substances may be used in combination. The combination of taste substances selected as the active ingredient is not particularly limited.

The “taste substance” may mean a substance involved in taste. Examples of the taste substance include a substance that exhibits a taste and a substance that imparts or enhances a taste. The “substance that exhibits a taste” may mean a substance that exhibits a taste by itself. The “substance that imparts or enhances a taste” may mean a substance that imparts a taste to a target or enhances the taste of a target, regardless of whether or not it exhibits a taste by itself. “Imparting a taste to a target” and “enhancing the taste of a target” may both mean that the taste of the target is increased. “Imparting a taste to a target” may mean, for example, imparting the taste to a target that does not exhibit a taste. “Enhancing the taste of a target” may mean, for example, enhancing the taste in a target that exhibits a taste. The type of taste is not particularly limited. Examples of the taste include sweet taste, salty taste, sour taste, bitter taste, umami, fatty taste, and kokumi. Examples of the substance that exhibits a taste include a substance that exhibits a sweet taste (also referred to as a “sweet substance”), a substance that exhibits a salty taste (also referred to as a “salty substance”), a substance that exhibits a sour taste (also referred to as a “sour substance”), a substance that exhibits a bitter taste (also referred to as a “bitter substance”), a substance that exhibits an umami taste (also referred to as an “umami substance”), and a substance that exhibits a fatty taste (also referred to as a “fatty taste substance”). Examples of the substance that imparts or enhances a taste include a substance that imparts or enhances a kokumi taste (also referred to as a “kokumi substance”).

Examples of the sweet substance include sugars, sugar alcohols, high-intensity sweeteners, and amino acids. Examples of the sugar that exhibits a sweet taste include monosaccharides such as glucose, fructose, galactose, and isomerized sugar; and disaccharides or higher oligosaccharides such as sucrose, lactose, maltose, trehalose, palatinose, lacto-fructo oligosaccharide, malto-oligosaccharide, isomalto-oligosaccharide, and fructo-oligosaccharide. Examples of the sugar alcohol that exhibits a sweet taste include erythritol, xylitol, sorbitol, mannitol, maltitol, and lactitol. Examples of the high-intensity sweetener (all of which exhibit a sweet taste) include aspartame (APM), acesulfame K (Ace-K), advantame, sucralose, neotame, and saccharin. Examples of the amino acid that exhibits a sweet taste include glycine, alanine, threonine, serine, glutamine, proline, and asparagine.

Examples of the sweet substance also include a component that activates a sweet taste receptor (also referred to as a “sweet taste receptor agonist”). Examples of the sweet taste receptor include T1R2/3. “T1R2/3” may mean a heterodimer of T1R2 and T1R3. For example, any of the sweet substances exemplified above can be a sweet taste receptor agonist. Specifically, for example, sucralose can be a T1R2/3 agonist.

Examples of the salty substance include inorganic salts. Examples of the inorganic salt that exhibits a salty taste include chlorides such as sodium chloride, potassium chloride, and ammonium chloride.

Examples of the salty substance also include a component that activates a salty taste receptor (also referred to as a “salty taste receptor agonist”). Examples of the salty taste receptor include ENaC (epithelial sodium channel), TMC4 (transmembrane channel-like 4), and TMC6 (transmembrane channel-like 6). For example, any of the salty substances exemplified above can be a salty taste receptor agonist.

Examples of the sour substance include organic acids, inorganic acids, and amino acids. Examples of the organic acid that exhibits a sour taste include acetic acid, lactic acid, malic acid, citric acid, tartaric acid, gluconic acid, succinic acid, and adipic acid. Examples of the inorganic acid that exhibits a sour taste include phosphoric acid. Examples of the amino acid that exhibits a sour taste include glutamic acid and aspartic acid.

Examples of the sour substance also include a component that activates a sour taste receptor (also referred to as a “sour taste receptor agonist”). Examples of the sour taste receptor include GPR91, PKD2L1, PKD1L3, and Otopetrin 1 (OTOP-1). For example, any of the sour substances exemplified above can be a sour taste receptor agonist. Specifically, for example, succinic acid can be a GPR91 agonist.

Examples of the bitter substance include amino acids, polyphenols, alkaloids, terpenoids, and inorganic salts. Examples of the amino acid that exhibits a bitter taste include branched-chain amino acids such as valine, leucine, and isoleucine; aromatic amino acids such as phenylalanine, tryptophan, and tyrosine; and other amino acids such as methionine, cysteine, lysine, arginine, histidine, and ornithine. Examples of the polyphenol that exhibits a bitter taste include catechin, naringin, and chlorogenic acid. Examples of the alkaloid that exhibits a bitter taste include caffeine, theobromine, and nicotine. Examples of the terpenoid that exhibits a bitter taste include humulone, cucurbitacin, momordicin, limonin, and bile acid. Examples of the inorganic salt that exhibits a bitter taste include potassium salts such as potassium chloride and magnesium salts such as magnesium chloride. Examples of the bitter substance also include other organic compounds such as diphenidol and salicin.

Examples of the bitter substance also include a component that activates a bitter taste receptor (also referred to as a “bitter taste receptor agonist”). Examples of the bitter taste receptor include T2R. Examples of T2R include human T2Rs such as hT2R1, hT2R2, hT2R3, hT2R5, hT2R7, hT2R8, hT2R9, hT2R10, hT2R13, hT2R14, hT2R16, hT2R38, hT2R39, hT2R40, hT2R41, hT2R43, hT2R44, hT2R45, hT2R46, hT2R47, hT2R48, hT2R49, hT2R50, and hT2R60; mouse T2Rs such as mT2R102, mT2R103, mT2R104, mT2R105, mT2R106, mT2R108, mT2R109, mT2R110, mT2R113, mT2R115, mT2R116, mT2R118, mT2R119, mT2R120, mT2R121, mT2R122, mT2R123, mT2R125, mT2R126, mT2R130, mT2R134, mT2R135, mT2R137, mT2R138, mT2R139, mT2R140, mT2R143, and mT2R144; bovine T2Rs such as bT2R3, bT2R4, bT2R5, bT2R8, bT2R10, bT2R12, bT2R16, bT2R31, bT2R38, bT2R39, bT2R40, bT2R41, bT2R46, bT2R55, bT2R60, and bT2R65, and bT2R109; and chicken T2Rs such as ggT2R1, ggT2R2, and ggT2R7. For example, any of the bitter substances exemplified above can be a bitter taste receptor agonist. Specifically, for example, diphenidol can be an agonist of hT2R1, hT2R4, hT2R7, hT2R10, hT2R13, hT2R14, hT2R16, hT2R38, hT2R39, hT2R40, hT2R43, hT2R44, hT2R46, hT2R47, hT2R49, and mT2R108. Specifically, for example, salicin can be an agonist of hT2R16 and mT2R126.

Examples of the umami substance include amino acids, nucleic acids, organic acids, amide compounds, and high-potency umami substances. Examples of the amino acid that exhibits an umami taste include glutamic acid and aspartic acid. Examples of the nucleic acid that exhibits an umami taste include inosinic acid, guanylic acid, and xanthylic acid. Examples of the organic acid that exhibits an umami taste include succinic acid. Examples of the amide compound that exhibits an umami taste include the amide compounds described in WO2014/208751 (specifically, a compound having two amide bonds). Examples of the amide compound described in WO2014/208751 include ATF100 (N—(R)-1-(2-(pyridin-2-yl)ethylcarbamoyl)-2-(benzyloxy)ethyl-4-methoxy-3-methylbenzamide). The “high-potency umami substance” may mean a substance having a taste potency of 50 times or more that of monosodium glutamate (MSG) (WO2013/133404). For example, a substance having a minimum concentration (threshold) at which an umami taste can be recognized of 0.00024% (w/w) or less in a human taste test is a high-potency umami substance. Further, for example, a substance having a concentration that exhibits an umami taste of a strength equal to that of a 0.5% (w/w) aqueous solution of monosodium glutamate of 0.01% (w/w) or less in a human taste test is a high-potency umami substance. Examples of the high-potency umami substance (all of which exhibit an umami taste) include amides described in WO2005/015158A2, WO2005/041684A2, and US2006-0045953A1; compounds comprising linked organic moieties described in WO2006/084184A2; compounds comprising linked heteroaryl moieties described in WO2006/084186A2; isosorbide derivatives described in US2009-0220662A1; alkyldienamides described in US2004-0202619A1 and US2004-0202760A1; saturated and unsaturated N-alkamides described in US2006-0057268A1 and US2006-0068071A1; oxalamides described in WO2011/095533A1; and compounds described in WO2011/020908A1, WO2011/004016A1, WO2010/094679A1, WO2007/027095A1, WO2008/046895A1, US2009-0110796A1, WO2007/124152A2, US2009-0311401A1, EP2168442A2, WO2006/009425, and EP1989944A1.

Examples of the umami substance also include a component that activates an umami taste receptor (also referred to as an “umami taste receptor agonist”). Examples of the umami taste receptor include T1R1/3. “T1R1/3” may mean a heterodimer of T1R1 and T1R3. For example, any of the umami substances exemplified above can be an umami taste receptor agonist. Specifically, for example, ATF100 can be a T1R1/3 agonist.

Examples of the fatty taste substance include fatty acids. Examples of the fatty acid that exhibits a fatty taste include C8 to C24 fatty acids. Examples of the fatty acid that exhibits a fatty taste (specifically, a C8 to C24 fatty acid) include caprylic acid (8:0), capric acid (10:0), lauric acid (12:0), myristic acid (14:0), palmitic acid (16:0), stearic acid (18:0), arachidic acid (20:0), behenic acid (22:0), lignoceric acid (24:0), palmitoleic acid (16:1), oleic acid (18:1), linoleic acid (18:2), linolenic acid (18:3), eicosenoic acid (20:1), erucic acid (22:1), arachidonic acid (20:4), eicosapentaenoic acid (EPA; 20:5), and docosahexaenoic acid (DHA; 22:6) (the numbers in parentheses indicate the number of carbon atoms and the number of double bonds).

Examples of the fatty taste substance also include a component that activates a fatty acid receptor (also referred to as a “fatty acid receptor agonist”). Examples of the fatty acid receptor include GPR40, GPR41, GPR43, GPR83, and GPR120. For example, any of the fatty taste substances exemplified above can be a fatty acid receptor agonist. Examples of the fatty acid receptor agonist such as a GPR120 agonist include 4-[(4-Fluoro-4′-methyl[1,1′-biphenyl]-2-yl) methoxy]-benzenepropanoic acid, 2-[3-fluoro-(pyridin-3-yloxy)phenyl]-1,2-benzisothiazol-3 (2H)-one-1,1-dioxide, and TUG-891 (3-[4-[[5-fluoro-2-(4-methylphenyl)phenyl]methoxy]phenyl]propanoic acid).

Examples of the kokumi substance include γ-glutamyl peptides. Examples of the γ-glutamyl peptide include γ-glutamyl tripeptides represented by the general formula: γ-Glu-X-Gly (wherein X represents an amino acid or an amino acid derivative) and γ-glutamyl dipeptides represented by the general formula: γ-Glu-Y (wherein Y represents an amino acid or an amino acid derivative). In the above general formulas, “γ-” means that X or Y is bonded via the γ-position carboxyl group of glutamic acid. As the γ-glutamyl peptide, one type of γ-glutamyl peptide may be used, or two or more types of γ-glutamyl peptides may be used in combination.

Specific examples of the amino acid selected as X and Y include neutral amino acids such as Gly, Ala, Val, Leu, Ile, Ser, Thr, Cys, Met, Asn, Gln, Pro, and Hyp; acidic amino acids such as Asp and Glu; basic amino acids such as Lys, Arg, and His; aromatic amino acids such as Phe, Tyr, and Trp; and other amino acids such as Orn, Sar, Cit, Nva, Nle, Abu, Tau, Hyp, t-Leu, Cle, Aib, Pen, and Hse.

In the present invention, the abbreviations for amino acid residues mean the following amino acids.
(1) Gly: Glycine
(2) Ala: Alanine
(3) Val: Valine
(4) Leu: Leucine
(5) Ile: Isoleucine
(6) Met: Methionine
(7) Phe: Phenylalanine
(8) Tyr: Tyrosine
(9) Trp: Tryptophan
(10) His: Histidine
(11) Lys: Lysine
(12) Arg: Arginine
(13) Ser: Serine
(14) Thr: Threonine
(15) Asp: Aspartic acid
(16) Glu: Glutamic acid
(17) Asn: Asparagine
(18) Gln: Glutamine
(19) Cys: Cysteine
(20) Pro: Proline
(21) Orn: Ornithine
(22) Sar: Sarcosine
(23) Cit: Citrulline
(24) Nva: Norvaline
(25) Nle: Norleucine
(26) Abu: α-aminobutyric acid
(27) Tau: Taurine
(28) Hyp: Hydroxyproline
(29) t-Leu: tert-leucine
(30) Cle: Cycloleucine
(31) Aib: α-aminoisobutyric acid (also known as: 2-methylalanine)
(32) Pen: Penicillamine
(33) Hse: Homoserine

An amino acid derivative refers to various derivatives of the amino acids as described above. Examples of amino acid derivatives include special amino acids, non-natural amino acids, amino alcohols, and amino acids in which one or more functional groups such as a terminal carbonyl group, a terminal amino group, and a thiol group of cysteine are substituted with various substituents. Specific examples of substituents include an alkyl group, an acyl group, a hydroxyl group, an amino group, an alkylamino group, a nitro group, a sulfonyl group, and various protecting groups. Specific examples of amino acid derivatives include Arg(NO₂): N-γ-nitroarginine, Cys(SNO): S-nitrosocysteine, Cys(S-Me): S-methylcysteine, Cys(S-allyl): S-allylcysteine, Val-NH₂: valine amide, Val-ol: valinol (also known as: 2-amino-3-methyl-1-butanol), Met(O): methionine sulfoxide, and Cys(S-Me)(O): S-methylcysteine sulfoxide.

Specific examples of the γ-glutamyl peptide include γ-Glu-Val-Gly, γ-Glu-Cys-Gly, γ-Glu-Abu-Gly, γ-Glu-Nva-Gly, γ-Glu-Abu, and γ-Glu-Nva.

The amino acids and amino acid derivatives constituting the γ-glutamyl peptide are all in the L-form unless otherwise specified.

Examples of the kokumi substance also include components that activate the calcium-sensing receptor (CaSR) (also referred to as “CaSR agonists”). For example, any of the kokumi substances exemplified above may be a CaSR agonist. Further, examples of CaSR agonists include cations such as calcium and gadolinium; basic peptides such as polyarginine and polylysine; polyamines such as putrescine, spermine, and spermidine;

proteins such as protamine; amino acids such as phenylalanine; cinacalcet; and aromatic components such as methional and isovaleraldehyde.

Examples of the taste substance also include components that activate a succinate receptor (also referred to as “succinate receptor agonists”). An example of the succinate receptor is GPR91. An example of a succinate receptor agonist, such as a GPR91 agonist, is succinic acid. Succinic acid can be, for example, a sour substance. That is, a succinate receptor such as GPR91 can also be, for example, a sour taste receptor.

Examples of the taste substance also include components that activate the GABA-B receptor (also referred to as “GABA-B receptor agonists”). Examples of the GABA-B receptor agonist include GABA (γ-aminobutyric acid) and SKF97541 (3-Aminopropyl(methyl)phosphinic acid). A GABA-B receptor agonist may have the function of enhancing the taste of other taste substances or targets containing them. For example, GABA can enhance salty taste.

Examples of the taste substance include components that activate a taste receptor (also referred to as “taste receptor agonists”). Examples of taste receptors include the sweet taste receptor, salty taste receptor, sour taste receptor, bitter taste receptor, umami taste receptor, fatty acid receptor, CaSR, succinate receptor, and GABA-B receptor. The sweet taste receptor, salty taste receptor, sour taste receptor, bitter taste receptor, umami taste receptor, fatty acid receptor, CaSR, succinate receptor, and GABA-B receptor, as well as their agonists, are as described above.

Whether a substance activates a receptor can be confirmed, for example, by bringing the receptor and the substance into contact and measuring the activation of the receptor due to contact with the substance. The contact between the receptor and the substance and the measurement of receptor activation thereby can be carried out with reference to, for example, screening methods for taste substances using the response of the receptor as an index (e.g., JP 2018-014999 A and WO 2011/081186). The receptor may be used, for example, by being supported on cells such as animal cells. When a receptor is used by being supported on a cell, “activation of the receptor” may mean the activation of the cell supporting the receptor. A cell supporting a receptor can be obtained, for example, by introducing a gene encoding the receptor into a cell and causing it to be expressed. In the case of a receptor that functions by forming a hetero-complex, a cell supporting the receptor can be obtained, for example, by introducing genes respectively encoding the subunits constituting the hetero-complex into a cell and co-expressing them. The activation of the receptor can be measured, for example, using an increase in cell membrane current or an increase in intracellular calcium ion concentration as an index (e.g., JP 2018-014999 A and WO 2011/081186). That is, for example, if the cell membrane current or intracellular calcium ion concentration increases under the condition where the receptor and the substance are brought into contact, as compared to the condition where they are not, it can be determined that the substance has activated the receptor. Methods for measuring the cell membrane current include the patch-clamp method and the voltage-clamp method. A method for measuring the intracellular calcium ion concentration is calcium imaging using a calcium indicator.

The amino acid selected as the taste substance may be a D-form, an L-form, or a combination thereof, as long as the object of the present invention can be achieved (e.g., a differentiation-promoting effect is obtained). The ratio of the D-form to the L-form in the combination is not particularly limited. The ratio of the D-form or the L-form in the combination may be, for example, in a molar ratio of 20-80%, 30-70%, 40-60%, or 45-55%. The amino acid selected as the taste substance may in particular be the L-form. It should be noted that when a D-form or L-form amino acid is selected, it is sufficient that the D-form or L-form of said amino acid is used as the active ingredient, and this does not preclude the further combined use of the L-form or D-form of said amino acid.

Examples of the taste substance particularly include a sweet substance, a salty substance, a sour substance, a bitter substance, an umami substance, a fatty taste substance, a kokumi substance, a succinate receptor agonist, and a GABA-B receptor agonist.

More particular examples of the taste substance include a sweet substance, a sour substance, a bitter substance, an umami substance, a fatty taste substance, a kokumi substance, a succinate receptor agonist, and a GABA-B receptor agonist.

Examples of the taste substance particularly include a sweet taste receptor agonist, a salty taste receptor agonist, a sour taste receptor agonist, a bitter taste receptor agonist, an umami taste receptor agonist, a fatty acid receptor agonist, a CaSR agonist, a succinate receptor agonist, and a GABA-B receptor agonist.

More particular examples of the taste substance include a sweet taste receptor agonist, a sour taste receptor agonist, a bitter taste receptor agonist, an umami taste receptor agonist, a fatty acid receptor agonist, a CaSR agonist, a succinate receptor agonist, and a GABA-B receptor agonist.

Examples of the taste substance particularly include ATF100, sucralose, cinacalcet, TUG-891, diphenidol, salicin, SKF97541, and succinic acid.

In one embodiment, examples of the taste substance particularly include taste substances other than ATF100, sucralose, cinacalcet, TUG-891, diphenidol, SKF97541, and succinic acid. For example, when the animal cell is a cell of an avian species such as a chicken, examples of the taste substance particularly include taste substances other than ATF100, sucralose, cinacalcet, TUG-891, diphenidol, SKF97541, and succinic acid. Examples of taste substances other than ATF100, sucralose, cinacalcet, TUG-891, diphenidol, SKF97541, and succinic acid include those among the taste substances exemplified above other than ATF100, sucralose, cinacalcet, TUG-891, diphenidol, SKF97541, and succinic acid. Examples of taste substances other than ATF100, sucralose, cinacalcet, TUG-891, diphenidol, SKF97541, and succinic acid particularly include those among sweet substances, salty substances, sour substances, bitter substances, umami substances, fatty taste substances, kokumi substances, succinate receptor agonists, and GABA-B receptor agonists, other than ATF100, sucralose, cinacalcet, TUG-891, diphenidol, SKF97541, and succinic acid. More particular examples of taste substances other than ATF100, sucralose, cinacalcet, TUG-891, diphenidol, SKF97541, and succinic acid include those among sweet substances, sour substances, bitter substances, umami substances, fatty taste substances, kokumi substances, succinate receptor agonists, and GABA-B receptor agonists, other than ATF100, sucralose, cinacalcet, TUG-891, diphenidol, SKF97541, and succinic acid. Examples of taste substances other than ATF100, sucralose, cinacalcet, TUG-891, diphenidol, SKF97541, and succinic acid particularly include those among sweet taste receptor agonists, salty taste receptor agonists, sour taste receptor agonists, bitter taste receptor agonists, umami taste receptor agonists, fatty acid receptor agonists, CaSR agonists, succinate receptor agonists, and GABA-B receptor agonists, other than ATF100, sucralose, cinacalcet, TUG-891, diphenidol, SKF97541, and succinic acid. More particular examples of taste substances other than ATF100, sucralose, cinacalcet, TUG-891, diphenidol, SKF97541, and succinic acid include those among sweet taste receptor agonists, sour taste receptor agonists, bitter taste receptor agonists, umami taste receptor agonists, fatty acid receptor agonists, CaSR agonists, succinate receptor agonists, and GABA-B receptor agonists, other than ATF100, sucralose, cinacalcet, TUG-891, diphenidol, SKF97541, and succinic acid. A particular example of a taste substance other than ATF100, sucralose, cinacalcet, TUG-891, diphenidol, SKF97541, and succinic acid is salicin.

In one embodiment, examples of the taste substance particularly include taste substances other than sugars, amino acids, organic acids, and calcium. In other words, in one embodiment, cases where the active ingredient consists of one or more taste substances selected from the group consisting of sugars, amino acids, organic acids, and calcium may be excluded from the present invention. Examples of taste substances other than sugars, amino acids, organic acids, and calcium include those among the taste substances exemplified above other than sugars, amino acids, organic acids, and calcium. Examples of taste substances other than sugars, amino acids, organic acids, and calcium particularly include those among sweet substances, salty substances, sour substances, bitter substances, umami substances, fatty taste substances, kokumi substances, succinate receptor agonists, and GABA-B receptor agonists, other than sugars, amino acids, organic acids, and calcium. More particular examples of taste substances other than sugars, amino acids, organic acids, and calcium include those among sweet substances, sour substances, bitter substances, umami substances, fatty taste substances, kokumi substances, succinate receptor agonists, and GABA-B receptor agonists, other than sugars, amino acids, organic acids, and calcium. Examples of taste substances other than sugars, amino acids, organic acids, and calcium particularly include those among sweet taste receptor agonists, salty taste receptor agonists, sour taste receptor agonists, bitter taste receptor agonists, umami taste receptor agonists, fatty acid receptor agonists, CaSR agonists, succinate receptor agonists, and GABA-B receptor agonists, other than sugars, amino acids, organic acids, and calcium. More particular examples of taste substances other than sugars, amino acids, organic acids, and calcium include those among sweet taste receptor agonists, sour taste receptor agonists, bitter taste receptor agonists, umami taste receptor agonists, fatty acid receptor agonists, CaSR agonists, succinate receptor agonists, and GABA-B receptor agonists, other than sugars, amino acids, organic acids, and calcium.

In one embodiment, examples of the taste substance particularly include taste substances other than glucose, amino acids, organic acids, and calcium. In other words, in one embodiment, cases where the active ingredient consists of one or more taste substances selected from the group consisting of glucose, amino acids, organic acids, and calcium may be excluded from the present invention. Examples of taste substances other than glucose, amino acids, organic acids, and calcium include those among the taste substances exemplified above other than glucose, amino acids, organic acids, and calcium. Examples of taste substances other than glucose, amino acids, organic acids, and calcium particularly include those among sweet substances, salty substances, sour substances, bitter substances, umami substances, fatty taste substances, kokumi substances, succinate receptor agonists, and GABA-B receptor agonists, other than glucose, amino acids, organic acids, and calcium. More particular examples of taste substances other than glucose, amino acids, organic acids, and calcium include those among sweet substances, sour substances, bitter substances, umami substances, fatty taste substances, kokumi substances, succinate receptor agonists, and GABA-B receptor agonists, other than glucose, amino acids, organic acids, and calcium. Examples of taste substances other than glucose, amino acids, organic acids, and calcium particularly include those among sweet taste receptor agonists, salty taste receptor agonists, sour taste receptor agonists, bitter taste receptor agonists, umami taste receptor agonists, fatty acid receptor agonists, CaSR agonists, succinate receptor agonists, and GABA-B receptor agonists, other than glucose, amino acids, organic acids, and calcium. More particular examples of taste substances other than glucose, amino acids, organic acids, and calcium include those among sweet taste receptor agonists, sour taste receptor agonists, bitter taste receptor agonists, umami taste receptor agonists, fatty acid receptor agonists, CaSR agonists, succinate receptor agonists, and GABA-B receptor agonists, other than glucose, amino acids, organic acids, and calcium.

Furthermore, the medium components described later may include those that correspond to taste substances. In one embodiment, examples of the taste substance particularly include taste substances other than the medium components described later. In other words, in one embodiment, cases where the active ingredient consists of the medium components described later may be excluded from the present invention. Examples of taste substances other than the medium components described later include those among the taste substances exemplified above other than the medium components described later. Examples of taste substances other than the medium components described later particularly include those among sweet substances, salty substances, sour substances, bitter substances, umami substances, fatty taste substances, kokumi substances, succinate receptor agonists, and GABA-B receptor agonists, other than the medium components described later. More particular examples of taste substances other than the medium components described later include those among sweet substances, sour substances, bitter substances, umami substances, fatty taste substances, kokumi substances, succinate receptor agonists, and GABA-B receptor agonists, other than the medium components described later. Examples of taste substances other than the medium components described later particularly include those among sweet taste receptor agonists, salty taste receptor agonists, sour taste receptor agonists, bitter taste receptor agonists, umami taste receptor agonists, fatty acid receptor agonists, CaSR agonists, succinate receptor agonists, and GABA-B receptor agonists, other than the medium components described later. More particular examples of taste substances other than the medium components described later include those among sweet taste receptor agonists, sour taste receptor agonists, bitter taste receptor agonists, umami taste receptor agonists, fatty acid receptor agonists, CaSR agonists, succinate receptor agonists, and GABA-B receptor agonists, other than the medium components described later.

When the active ingredient can form a salt, the active ingredient may be used as a free form, as a salt, or as a combination thereof. That is, the term “active ingredient”, unless otherwise specified, may mean the active ingredient in its free form, a salt thereof, or a combination thereof. The “free form” means a form that has not formed a salt. Also, when the active ingredient can form a hydrate, the active ingredient may be used as a non-hydrate, as a hydrate, or as a combination thereof. That is, the term “active ingredient” (e.g., “active ingredient in its free form” or “salt of the active ingredient”), unless otherwise specified, may include non-hydrates and hydrates. The active ingredient may take any form, such as an ion, at the time of use.

The salt is not particularly limited as long as it is acceptable for the purposes of the present invention. For example, when producing an edible product by culturing animal cells, an orally acceptable salt may be selected. For example, specific examples of salts for acidic groups such as a carboxyl group include ammonium salts; salts with alkali metals such as sodium and potassium; salts with alkaline earth metals such as calcium and magnesium; aluminum salts; zinc salts; salts with organic amines such as triethylamine, ethanolamine, morpholine, pyrrolidine, piperidine, piperazine, and dicyclohexylamine; and salts with basic amino acids such as arginine and lysine. Furthermore, for example, specific examples of salts for basic groups such as an amino group include salts with inorganic acids such as hydrochloric acid, sulfuric acid, phosphoric acid, nitric acid, and hydrobromic acid; salts with organic carboxylic acids such as acetic acid, citric acid, benzoic acid, maleic acid, fumaric acid, tartaric acid, succinic acid, tannic acid, butyric acid, hybenzic acid, pamoic acid, enanthic acid, decanoic acid, theoclic acid, salicylic acid, lactic acid, oxalic acid, mandelic acid, malic acid, methylmalonic acid, and adipic acid; and salts with organic sulfonic acids such as methanesulfonic acid, benzenesulfonic acid, and p-toluenesulfoni2c acid. For example, the salt of succinic acid is particularly a sodium salt. As the salt, one type of salt may be used, or two or more types of salts may be used in combination.

As the active ingredient, a commercially available product may be used, or one that has been appropriately manufactured and obtained may be used. The method for manufacturing the active ingredient is not particularly limited. The active ingredient can be manufactured, for example, by chemical synthesis, enzymatic reaction, a fermentation method, an extraction method, or a combination thereof. The active ingredient may or may not be purified to a desired degree. That is, as the active ingredient, a purified product may be used, or a material containing the active ingredient may be used. As the active ingredient, for example, a material with a content of the active ingredient of 1% (w/w) or more, 5% (w/w) or more, 10% (w/w) or more, 30% (w/w) or more, 50% (w/w) or more, 70% (w/w) or more, 90% (w/w) or more, or 95% (w/w) or more may be used.

The amount of the active ingredient (e.g., content (concentration) or amount used) shall, when using a material containing the active ingredient, be calculated based on the amount of the active ingredient itself in said material. Furthermore, the amount of the active ingredient (e.g., content (concentration) or amount used) shall, when the active ingredient forms a salt or hydrate, be calculated based on a value obtained by converting the mass of the salt or hydrate to the mass of the equimolar non-hydrated free form.

### <2> Composition of the Present Invention

The composition of the present invention is a composition containing an active ingredient (i.e., a taste substance).

The composition of the present invention can be used, for example, for culturing animal cells. That is, the composition of the present invention may be a composition for animal cell culture. The composition of the present invention can be used for culturing animal cells, for example, in the manner described in the method of the present invention described later.

The composition of the present invention (specifically, a composition for animal cell culture) may be, for example, a medium. Examples of the medium include a basal medium, a fed-batch medium (feed medium), and a perfusion medium. The “basal medium” may mean the medium at the start of culturing. The basal medium is also referred to as an “initial medium”. The “fed-batch medium” may mean a medium that is supplied to the culture system after the start of culturing in fed-batch culture. The “perfusion medium” may mean a medium that is supplied to the culture system after the start of culturing in continuous culture (which is not limited to perfusion culture).

The composition of the present invention (specifically, a composition for animal cell culture) may be, for example, a medium supplement. A “medium supplement” may mean a composition that is used by being added to a medium. Examples of the medium to which the medium supplement is added include a basal medium, a fed-batch medium, and a perfusion medium.

By using the composition of the present invention, the differentiation of animal cells may be promoted, that is, an effect of promoting the differentiation of animal cells (a differentiation-promoting effect) may be obtained. For example, a differentiation-promoting effect may be obtained by culturing animal cells in the composition of the present invention that is a medium. Furthermore, for example, a differentiation-promoting effect may be obtained by culturing animal cells in a medium to which the composition of the present invention, which is a medium supplement, has been added. In other words, the composition of the present invention may have a function of promoting the differentiation of animal cells (a differentiation-promoting function). Therefore, the composition of the present invention may be used for promoting the differentiation of animal cells. That is, the composition of the present invention may be, for example, a composition for promoting the differentiation of animal cells.

In one embodiment, a product (e.g., cultured meat) may be manufactured by culturing animal cells. When a product is manufactured by culturing animal cells, the use of the composition of the present invention may improve the production of the product. For example, the promotion of animal cell differentiation by using the composition of the present invention may improve the production of the product. That is, one embodiment of the composition of the present invention (specifically, a composition for animal cell culture) may be a composition for improving the production of a product.

The composition of the present invention may consist of the active ingredient, or it may contain components other than the active ingredient. Components other than the active ingredient are also referred to as “additional components”. That is, the active ingredient may be used as the composition of the present invention as it is, or in combination with additional components. As the additional components, one type of component may be used, or two or more types of components may be used in combination.

That is, the present invention provides a method for producing the composition of the present invention, which includes using the active ingredient. The mode of use of the active ingredient in the production of the composition of the present invention is not particularly limited as long as a composition containing the active ingredient is obtained. The use of the active ingredient in the production of the composition of the present invention includes incorporating the active ingredient into the composition. “Incorporating the active ingredient into the composition” may mean causing the composition to contain the active ingredient. “Incorporating the active ingredient into the composition” may specifically mean combining the active ingredient with additional components.

The additional components are not particularly limited as long as the object of the present invention can be achieved (e.g., a differentiation-promoting effect is obtained). The additional components can be appropriately selected, for example, according to various conditions such as the type of animal cell and the mode of use of the composition of the present invention. Examples of additional components include medium components. Medium components will be described later. For example, at least when the composition of the present invention is a medium, the composition of the present invention may contain medium components.

The composition of the present invention may, for example, be appropriately formulated. In formulation, additives may be used as appropriate. That is, examples of additional components also include additives used in formulation. Examples of additives include excipients, binders, disintegrants, lubricants, stabilizers, flavoring and odor-masking agents, diluents, and surfactants. The additives can be appropriately selected, for example, according to various conditions such as the shape of the composition of the present invention.

The shape of the composition of the present invention is not particularly limited. The composition of the present invention may be in any shape, for example, powder, flakes, tablets, paste, or liquid.

The content and content ratio of each component (i.e., the active ingredient and optional additional components) in the composition of the present invention are not particularly limited as long as the object of the present invention can be achieved (e.g., a differentiation-promoting effect is obtained). That is, for example, the content of the active ingredient in the composition of the present invention may be an effective amount (e.g., an amount that provides a differentiation-promoting effect). The content and content ratio of each component in the composition of the present invention can be appropriately set, for example, according to various conditions such as the type of animal cell and the mode of use of the composition of the present invention.

The content of the active ingredient in the composition of the present invention is more than 0% (w/w) and 100% (w/w) or less. The content of the active ingredient in the composition of the present invention may be, for example, 1 ppb (w/w) or more, 10 ppb (w/w) or more, 100 ppb (w/w) or more, 1 ppm (w/w) or more, 10 ppm (w/w) or more, 100 ppm (w/w) or more, 1000 ppm (w/w) or more, 1% (w/w) or more, 2% (w/w) or more, 5% (w/w) or more, or 10% (w/w) or more, and may be 100% (w/w) or less, less than 100% (w/w), 99.9% (w/w) or less, 90% (w/w) or less, 50% (w/w) or less, 20% (w/w) or less, 10% (w/w) or less, 5% (w/w) or less, 2% (w/w) or less, 1% (w/w) or less, 1000 ppm (w/w) or less, 100 ppm (w/w) or less, 10 ppm (w/w) or less, or 1 ppm (w/w) or less, or any non-contradictory combination thereof. Specifically, the content of the active ingredient in the composition of the present invention may be, for example, 1 ppb (w/w) to 1 ppm (w/w), 1 ppm (w/w) to 10 ppm (w/w), 10 ppm (w/w) to 100 ppm (w/w), 100 ppm (w/w) to 1000 ppm (w/w), 1000 ppm (w/w) to 1% (w/w), 1% (w/w) to 10% (w/w), or 10% (w/w) to 20% (w/w). Specifically, the content of the active ingredient in the composition of the present invention may be, for example, 1 ppb (w/w) to 10% (w/w), 1 ppb (w/w) to 1% (w/w), or 1 ppb (w/w) to 1000 ppm (w/w). When the composition of the present invention contains two or more active ingredients, the content of these two or more active ingredients in the composition of the present invention may be set, independently or in total, within the range of the content of the active ingredient in the composition of the present invention exemplified above (provided that the total content of these two or more active ingredients in the composition of the present invention is 100% (w/w) or less). When the composition of the present invention contains two or more active ingredients, “the content of the active ingredient in the composition of the present invention” shall, unless otherwise specified, mean the total content of these two or more active ingredients in the composition of the present invention.

In one embodiment, the composition of the present invention may be substantially free of serum. “The composition of the present invention being substantially free of serum” may mean that the serum content in the composition of the present invention is 1% (w/w) or less, 0.1% (w/w) or less, 0.01% (w/w) or less, or 0.001% (w/w) or less, and may also include the case where the serum content in the composition of the present invention is 0 (zero) (i.e., the composition of the present invention does not contain serum).

In one embodiment, the composition of the present invention may be substantially free of animal-derived albumin. “The composition of the present invention being substantially free of animal-derived albumin” may mean that the content of animal-derived albumin in the composition of the present invention is 1% (w/w) or less, 0.1% (w/w) or less, 0.01% (w/w) or less, or 0.001% (w/w) or less, and may also include the case where the content of animal-derived albumin in the composition of the present invention is 0 (zero) (i.e., the composition of the present invention does not contain animal-derived albumin).

The content of each component (i.e., the active ingredient and optional additional components) in the composition of the present invention can be set, for example, so as to obtain the concentration of each component in the medium in the method of the present invention described later.

When the composition of the present invention contains two or more components, these components may be mixed with each other and contained in the composition of the present invention, or they may be contained in the composition of the present invention separately, either individually or in any combination. For example, the composition of the present invention may be provided as a set of a package of the active ingredient and a package of the additional components. In such a case, the components included in the set can be used in combination as appropriate at the time of use.

### <3> Method of the Present Invention

The method of the present invention is a method for culturing animal cells, which includes a step of culturing animal cells in the presence of an active ingredient (i.e., a taste substance). This step is also referred to as the “culturing step”. The culturing step may specifically be a step of culturing animal cells in the presence of the active ingredient to differentiate the animal cells.

By using the active ingredient, specifically by culturing animal cells in the presence of the active ingredient, the differentiation of animal cells may be promoted, that is, an effect of promoting the differentiation of animal cells (a differentiation-promoting effect) may be obtained. That is, one embodiment of the method of the present invention (specifically, a method for culturing animal cells) may be, for example, a method for promoting the differentiation of animal cells. The purpose of culturing animal cells is not particularly limited.

In one embodiment, a product (e.g., cultured meat) may be produced by culturing animal cells. That is, when animal cells can form cultured meat through culturing, cultured meat can be produced by culturing said cells. The cultured meat may be mainly composed of muscle tissue. Therefore, “animal cells can form cultured meat through culturing” may mean, for example, that animal cells can differentiate and form muscle tissue through culturing, and specifically, it may mean that animal cells can differentiate into myotubes and form muscle tissue through culturing. That is, one embodiment of the method of the present invention (specifically, a method for culturing animal cells) may be a method for producing a product, including a step of culturing animal cells in the presence of an active ingredient. The culturing step may also specifically be a step of producing a product by culturing animal cells in the presence of an active ingredient. In the method for producing cultured meat, the cultured animal cells may form cultured meat. When producing a product by culturing animal cells, the use of the active ingredient may improve the production of the product. For example, promoting the differentiation of animal cells by using the active ingredient may improve the production of the product. That is, one embodiment of the method of the present invention (specifically, a method for culturing animal cells) may be a method for improving the production of a product.

“Animal cell” means a cell of an animal. The animal cell is not particularly limited as long as it has differentiation potential. The animal cell can be appropriately selected, for example, according to various conditions such as the purpose of culturing the animal cells. For example, when producing cultured meat by culturing animal cells, the animal cell is not particularly limited as long as it can form cultured meat through culturing. “Animal” may mean an organism classified in the kingdom Animalia. Examples of animals include vertebrates and aquatic organisms. Examples of vertebrates include mammals, birds, reptiles, amphibians, and fish. Vertebrates particularly include mammals, birds, and fish. Vertebrates particularly include mammals. Examples of mammals include primates such as humans, monkeys, and chimpanzees; rodents such as hamsters, mice, rats, and guinea pigs; other terrestrial mammals such as cows, pigs, sheep, goats, rabbits, horses, deer, water buffalos, reindeer, donkeys, camels, dogs, and cats; and aquatic mammals such as whales, dolphins, sea lions, and walruses. Examples of birds include chickens, turkeys, ducks, geese, guinea fowls, quails, and ostriches. Examples of fish include eels, tuna, longtooth groupers, sea breams, salmon, cod, and pufferfish. In addition to aquatic mammals and fish, examples of aquatic organisms include crustaceans such as shrimps and crabs, shellfish such as scallops and oysters, and other aquatic organisms such as squid and octopus. Aquatic organisms particularly include fish and crustaceans. Animals suitable for the production of cultured meat include animals for meat production. Animals for meat production include those among the animals exemplified above whose meat can be used for food. Animals for meat production (especially mammals) particularly include cows, pigs, sheep, goats, and rabbits. Animals for meat production (especially mammals) more particularly include cows and pigs. Animals for meat production (especially mammals) even more particularly include cows. Animals for meat production (especially birds) particularly include chickens, turkeys, ducks, geese, guinea fowls, quails, and ostriches. Animals for meat production (especially birds) more particularly include chickens. Animals for meat production (especially aquatic organisms) particularly include the fish and crustaceans exemplified above. Animals for meat production (especially aquatic organisms) more particularly include eels, tuna, and shrimps. The tissue or cell from which the animal cell is derived is not particularly limited. Examples of the tissue or cell from which the animal cell is derived include ovary, kidney, adrenal gland, tongue epithelium, olfactory epithelium, pineal body, thyroid, melanocyte, skin, spleen, liver, lung, pancreas, uterus, stomach, colon, small intestine, large intestine, bladder, prostate, testis, thymus, muscle, connective tissue, bone, cartilage, vascular tissue, blood (including umbilical cord blood), bone marrow, heart, eye, brain, and nervous tissue. The tissue or cell from which the animal cell is derived is particularly muscle. Specific examples of animal cells include somatic cells, stem cells, and progenitor cells. Examples of somatic cells include myoblasts and fibroblasts. Examples of stem cells include adult stem cells such as hematopoietic stem cells, satellite cells (muscle stem cells), neural stem cells, mesenchymal stem cells, mammary gland stem cells, olfactory mucosal stem cells, neural crest stem cells, liver stem cells, pancreatic stem cells, germline stem cells, intestinal stem cells, and hair follicle stem cells; pluripotent stem cells such as embryonic stem cells (ES cells), embryonic carcinoma cells, embryonic germ stem cells, and induced pluripotent stem cells (iPS cells); and cancer stem cells. Examples of progenitor cells include satellite cells, pancreatic progenitor cells, vascular progenitor cells, vascular endothelial progenitor cells, and hematopoietic progenitor cells (such as CD34-positive cells derived from umbilical cord blood). Animal cells suitable for the production of cultured meat include stem cells such as satellite cells (muscle stem cells), mesenchymal stem cells, embryonic stem cells (ES cells), and induced pluripotent stem cells (iPS cells), and myoblasts. Animal cells suitable for the production of cultured meat particularly include satellite cells (muscle stem cells) and myoblasts. Myoblasts may be classified as stem cells. The animal cells may, for example, express a taste receptor corresponding to the taste substance (i.e., a taste receptor activated by the taste substance).

“An animal cell is a cell that satisfies a certain condition” means that it is sufficient for the animal cell to satisfy said condition during at least a part of the culture period, and it is not required for the animal cell to satisfy said condition for the entire culture period. That is, “an animal cell is a cell that satisfies a certain condition” is not limited to cases where the animal cell satisfies said condition at the start of culturing (i.e., starting the culture using an animal cell that satisfies said condition), but may also include cases where the animal cell comes to satisfy said condition after the start of culturing. For example, “the animal cell is a myoblast” is not limited to cases where the culture is started using animal cells that are myoblasts, but may also include cases where the animal cells differentiate into myoblasts after the start of culturing.

The culturing of animal cells is carried out in the presence of the active ingredient. “The culturing of animal cells is carried out in the presence of the active ingredient” may mean, for example, culturing the animal cells in a medium containing the active ingredient. “The culturing of animal cells is carried out in the presence of the active ingredient” may also mean, for example, that the active ingredient is supplied to the culture system during the culturing of the animal cells.

The active ingredient may be used for culturing animal cells, for example, in the form of the composition of the present invention. The composition of the present invention may be used for culturing animal cells, for example, as a medium. That is, for example, when the composition of the present invention is a medium, the animal cells may be cultured in the composition of the present invention (i.e., the medium). That is, “culturing the animal cells in a medium containing the active ingredient” may include culturing the animal cells in the composition of the present invention that is a medium. The composition of the present invention may also be used for culturing animal cells, for example, as a medium supplement. That is, for example, when the composition of the present invention is a medium supplement, the composition of the present invention (i.e., the medium supplement) may be added to a medium, and the animal cells may be cultured in the medium to which the composition of the present invention has been added. That is, “culturing the animal cells in a medium containing the active ingredient” may include culturing the animal cells in a medium to which the composition of the present invention, which is a medium supplement, has been added. Also, for example, the composition of the present invention or a medium to which it has been added may be supplied to the culture system during the culturing of the animal cells. That is, “the active ingredient is supplied to the culture system during the culturing of the animal cells” may include that the composition of the present invention or a medium to which it has been added is supplied to the culture system during the culturing of the animal cells.

The medium composition and culture conditions are not particularly limited as long as the object of the present invention can be achieved (e.g., a differentiation-promoting effect is obtained), except that the culturing of animal cells is carried out in the presence of the active ingredient and the animal cells differentiate. The medium composition and culture conditions can be appropriately set, for example, according to various conditions such as the type of animal cell and the purpose of culturing the animal cells. For example, when producing cultured meat by culturing animal cells, the medium composition and culture conditions are not particularly limited as long as cultured meat can be formed by culturing the animal cells. The culturing of animal cells, except for being carried out in the presence of the active ingredient, can be performed, for example, using a normal medium and normal conditions used for culturing animal cells (specifically, for differentiation of animal cells) as they are, or by modifying them as appropriate. When producing cultured meat by culturing animal cells, the culturing of animal cells, except for being carried out in the presence of the active ingredient, can be performed, for example, using a normal medium and normal conditions used for the formation of muscle tissue by differentiation of animal cells (e.g., for the production of cultured meat) as they are, or by modifying them as appropriate.

The culturing can be carried out, for example, using a liquid medium. The culturing can be carried out by batch culture, fed-batch culture, continuous culture, or a combination thereof. Examples of continuous culture include perfusion culture and chemostat culture. The medium at the start of culturing is also referred to as an “initial medium” or “basal medium”. The medium supplied to the culture system (e.g., the initial medium) in fed-batch culture is also referred to as a “fed-batch medium (feed medium)”. The medium supplied to the culture system (e.g., the initial medium) in continuous culture (which is not limited to perfusion culture) is also referred to as a “perfusion medium”. Furthermore, supplying a feed medium or a perfusion medium to the culture system in fed-batch culture or continuous culture is also simply referred to as “medium supply”. Medium supply may be carried out throughout the entire culture period, or only during a part of the culture period. Medium supply may be carried out continuously or intermittently. During culturing (especially continuous culture such as perfusion culture), withdrawal of the culture medium may be carried out. The withdrawal of the culture medium may be carried out throughout the entire culture period, or only during a part of the culture period. The withdrawal of the culture medium may be carried out continuously or intermittently. The withdrawal of the culture medium and the medium supply may or may not be carried out simultaneously. The culture vessel may be coated with a cell adhesion molecule such as fibronectin, for example. The culturing may be carried out three-dimensionally, for example, using a scaffold material. When producing cultured meat by culturing animal cells, the culturing may be carried out three-dimensionally, for example, using a scaffold material shaped to match the shape of the cultured meat to be formed.

The medium used for culturing can be selected independently, for example, for the basal medium, the fed-batch medium, and the perfusion medium.

The medium used for culturing may be a commercially available medium or a medium prepared as appropriate.

Examples of the medium used for culturing include a medium containing components essential for culturing animal cells (e.g., a carbon source, a nitrogen source, inorganic salts, etc.).

Specific examples of the medium used for culturing include Dulbecco's Modified Eagle's Medium (DMEM), Ham's Nutrient Mixture F12, DMEM/F12 medium, McCoy's 5A medium, Minimum Essential Medium (MEM), Eagle's Minimum Essential Medium (EMEM), alpha Modified Eagle's Minimum Essential Medium (αMEM), Roswell Park Memorial Institute (RPMI) 1640 medium, Iscove's Modified Dulbecco's Medium (IMDM), MCDB131 medium, William's Medium E, and Fischer's Medium.

Specific examples of the medium used for culturing (which may be a medium used particularly for culturing stem cells (especially pluripotent stem cells)) include STEMPRO® hESC SFM medium (Life Technologies), mTeSR1 medium (STEMCELL Technologies), TeSR2 medium (STEMCELL Technologies), TeSR-E8 medium (STEMCELL Technologies), Essential 8 medium (Life Technologies), HEScGRO™ Serum-Free Medium for hES cells (Millipore), PluriSTEM™ Human ES/IPS Medium (EMD Millipore), NutriStem® hESC XF medium (Biological Industries Israel Beit-Haemek), NutriStem™ XF/FF Culture Medium (Stemgent), AF NutriStem® hESC XF medium (Biological Industries Israel Beit-Haemek), S-medium (DS Pharma Biomedical Co., Ltd.), StemFit® AK03N medium (Ajinomoto Co., Inc.), hESF9 medium, hESF-FX medium, CDM medium, DEF-CS 500 Xeno-Free 3D Spheroid Culture Medium (Cellartis), StemFlex medium (Thermo Fisher Scientific), and the like.

Other commercially available media include media for animal cell culture such as CELLIST Basal Media BASAL3, BASAL4P, BASAL10 (Ajinomoto Co., Inc.), Opti-MEM (Thermo Fisher Scientific), RPMI 1640 (Thermo Fisher Scientific), CD293 (Thermo Fisher Scientific), CHO-S-SFMII (Thermo Fisher Scientific), CHO-SF (Sigma-Aldrich), EX-CELL CD CHO (Sigma-Aldrich), EX-CELLTM302 (Sigma-Aldrich), IS CHO-CD (Irvine Scientific), and IS CHO-CDXP (Irvine Scientific).

The media exemplified above may be used for culturing, for example, by adding the active ingredient.

The medium used for culturing may be, for example, the composition of the present invention (specifically, the composition of the present invention that is a medium). That is, when the composition of the present invention is a medium, the composition of the present invention may be used for culturing as it is, or after being prepared as a liquid medium of a desired composition as appropriate. For example, the composition of the present invention may be diluted with an aqueous medium such as water or an aqueous buffer to be prepared as a liquid medium and used for culturing.

The medium used for culturing may be, for example, a medium to which the composition of the present invention (specifically, the composition of the present invention that is a medium supplement) has been added. The medium to which the composition of the present invention is added may be a commercially available medium or a medium prepared as appropriate.

The medium may contain various medium components. Examples of medium components include carbon sources, amino acid sources, peptides, proteins, vitamins, fatty acids, lipids, organic acids, nucleic acids, amines, antioxidants, inorganic components, pH buffering agents, growth factors, cytokines, hormones, cell adhesion factors, extracellular matrix components, serum, lecithin, pea-derived materials, yeast extract, antibiotics, and gene expression inducers. Any of these medium components may be, for example, essential or effective for the survival or proliferation of animal cells. Any of these medium components may, for example, be pre-contained in the media as exemplified above, or may be added to the media as exemplified above.

Examples of the carbon source include sugars such as glucose, fructose, sucrose, and maltose.

Examples of the amino acid source include amino acids. Peptides and proteins can both be examples of amino acid sources. Examples of amino acids include glycine, alanine, valine, leucine, isoleucine, cysteine, methionine, phenylalanine, tyrosine, tryptophan, histidine, lysine, arginine, serine, threonine, aspartic acid, glutamic acid, asparagine, glutamine, proline, and ornithine. The amino acids may be, for example, in the L-form.

Examples of peptides include dipeptides and tripeptides. Specific examples of peptides include glycyl-glycyl-glycine and soy peptide. The description concerning amino acids can be applied mutatis mutandis to the amino acids constituting the peptides.

Examples of proteins include albumin and transferrin.

Examples of vitamins include vitamin A, vitamin B1, vitamin B2, vitamin B3, vitamin B5, vitamin B6, vitamin B7, vitamin B9, vitamin B12, vitamin C, vitamin D, vitamin E, and vitamin K, and their precursors.

Examples of fatty acids include oleic acid, arachidonic acid, and linoleic acid.

An example of a lipid is cholesterol.

An example of an organic acid is pyruvic acid (such as sodium pyruvate).

Examples of nucleic acids include hypoxanthine and thymidine.

An example of an amine is a polyamine such as putrescine.

An example of an antioxidant is lipoic acid.

Examples of inorganic components include sodium, potassium, calcium, magnesium, phosphorus, and various trace elements (e.g., Co, Cu, F, Fe, Mn, Mo, Ni, Se, Si, Ni, Bi, V, and Zn). Specific examples of inorganic components include inorganic salts such as sodium chloride, potassium chloride, calcium chloride, magnesium sulfate, and sodium dihydrogen phosphate.

Examples of pH buffering agents include sodium bicarbonate, phosphate, N, N-bis(2-hydroxyethyl)-2-aminoethanesulfonic acid (BES), 4-(2-hydroxyethyl)-1-piperazineethanesulfonic acid (HEPES), and N-[tris(hydroxymethyl)methyl]glycine (Tricine).

Examples of growth factors include fibroblast growth factor (FGF), hepatocyte growth factor (HGF), epidermal growth factor (EGF), transforming growth factor (TGF)-α, transforming growth factor (TGF)-β, vascular endothelial growth factor (VEGF), activin A, and insulin-like growth factor-1 (IGF-1). An example of FGF is basic fibroblast growth factor (bFGF). A particular example of a growth factor is bFGF. That is, as a medium component, at least bFGF may be used. That is, the medium may contain at least bFGF. An example of bFGF is bovine basic fibroblast growth factor (bbFGF). “Bovine basic fibroblast growth factor (bbFGF)” may mean bFGF that is bovine-derived. “Bovine” in relation to bbFGF may mean an organism of the genus Bos. An example of an organism of the genus Bos is Bos taurus. bbFGF is not limited to bFGF found in organisms of the genus Bos, and may be a variant thereof.

An example of a cytokine is an interleukin.

Examples of hormones include dexamethasone, hydrocortisone, estradiol, progesterone, glucagon, and insulin.

Examples of cell adhesion factors or extracellular matrix components include Type I collagen, Type II collagen, fibronectin, laminin, poly-L-lysine, and poly-D-lysine.

Examples of antibiotics include amphotericin B, kanamycin, gentamicin, streptomycin, and penicillin.

“Pea-derived material” means a material derived from peas. An example of a pea-derived material is a processed pea product. Examples of pea-derived materials (e.g., processed pea products) include fermented pea products and pea protein.

When a medium component that corresponds to a taste substance is used for culturing, said medium component may or may not be selected as the active ingredient.

In one embodiment, the medium may be substantially free of serum. The medium may be substantially free of serum, for example, for the entire duration of the culture. “A medium being substantially free of serum” may mean that the serum content in the medium is 1% (w/w) or less, 0.1% (w/w) or less, 0.01% (w/w) or less, or 0.001% (w/w) or less, and may also include cases where the serum content in the medium is 0 (zero) (i.e., the medium does not contain serum).

In one embodiment, the medium may be substantially free of animal-derived albumin. Examples of the animal from which the albumin is derived include the animals exemplified for the animal cells. The medium may be substantially free of animal-derived albumin, for example, for the entire duration of the culture. “A medium being substantially free of animal-derived albumin” may mean that the content of animal-derived albumin in the medium is 1% (w/w) or less, 0.1% (w/w) or less, 0.01% (w/w) or less, or 0.001% (w/w) or less, and may also include cases where the content of animal-derived albumin in the medium is 0 (zero) (i.e., the medium does not contain animal-derived albumin).

Various components such as the active ingredient may each be contained in the initial medium, the fed-batch medium, the perfusion medium, or a combination thereof. That is, during the culturing process, various components such as the active ingredient may be supplied to the medium, either alone or in any combination. Any of these components may be supplied once or multiple times, or may be supplied continuously. The composition (e.g., the types and/or concentrations of the components contained) of the initial medium, the fed-batch medium, and the perfusion medium may or may not be the same. That is, the types of components contained in the initial medium may or may not be the same as the types of components contained in the fed-batch or perfusion medium. Furthermore, the concentration of each component contained in the initial medium may or may not be the same as the concentration of each component contained in the fed-batch or perfusion medium. For example, when a fed-batch medium is used in perfusion culture, the composition of the initial medium and the fed-batch medium may be the same. Also, two or more fed-batch or perfusion media with different compositions (e.g., types and/or concentrations of components contained) may be used. For example, when multiple supplies of the fed-batch or perfusion medium are performed intermittently, the composition of the fed-batch or perfusion medium may or may not be the same each time. Also, various components such as the active ingredient may each be supplied to the medium in a form not contained in the fed-batch or perfusion medium, such as a powder.

The seeding amount of animal cells at the start of culturing may be, for example, in terms of viable cell count, 1×10² cells/mL or more, 1×10³ cells/mL or more, 1×10⁴ cells/mL or more, 1×10⁵ cells/mL or more, 1×10⁶ cells/mL or more, or 1×10⁷ cells/mL or more, and may be 1×10⁸ cells/mL or less, 1×10⁷ cells/mL or less, 1×10⁶ cells/mL or less, 1×10⁵ cells/mL or less, 1×10⁴ cells/mL or less, or 1×10³ cells/mL or less, or any non-contradictory combination thereof. Specifically, the seeding amount of animal cells at the start of culturing may be, for example, in terms of viable cell count, 1×10² to 1×10³ cells/mL, 1×10³ to 1×10⁴ cells/mL, 1×10⁴ to 1×10⁵ cells/mL, 1×10⁵ to 1×10⁶ cells/mL, 1×10⁶ to 1×10⁷ cells/mL, or 1×10⁷ to 1×10⁸ cells/mL. Specifically, the seeding amount of animal cells at the start of culturing may be, for example, in terms of viable cell count, 1×10² to 1×10⁸ cells/mL, 1×10³ to 1×10⁷ cells/mL, or 1×10³ to 1×10⁶ cells/mL. The viable cell count can be measured, for example, using a Vi-CELL™ XR Cell Viability Analyzer (Beckman Coulter, Inc.).

The culturing may be carried out, for example, in a CO₂-containing atmosphere, such as 5-15% CO₂. The pH of the medium may be, for example, around neutral. “Around neutral” may mean, for example, a pH of 6-8, a pH of 6.5-7.5, or a pH of 6.8-7.2. During culturing, the pH of the medium can be adjusted as necessary. The pH of the medium can be adjusted using various alkaline or acidic substances such as ammonia gas, aqueous ammonia, sodium carbonate, sodium bicarbonate, potassium carbonate, potassium bicarbonate, magnesium carbonate, sodium hydroxide, potassium hydroxide, calcium hydroxide, and magnesium hydroxide. The culture temperature may be, for example, 30-38° C. The culture period may be, for example, 0.5 days or more, 1 day or more, 2 days or more, 3 days or more, 4 days or more, 5 days or more, 6 days or more, 7 days or more, 8 days or more, 9 days or more, 10 days or more, 12 days or more, 15 days or more, or 20 days or more, and may be 60 days or less, 50 days or less, 40 days or less, 30 days or less, 25 days or less, 20 days or less, 15 days or less, 12 days or less, 10 days or less, 9 days or less, 8 days or less, or 7 days or less, or any non-contradictory combination thereof. Specifically, the culture period may be, for example, 1-60 days, 3-25 days, or 5-20 days. The culturing may be continued, for example, until the product is produced to a desired extent.

The amount used (e.g., concentration in the medium) and the ratio used (e.g., concentration ratio in the medium) of each component (e.g., medium components such as the active ingredient) in the method of the present invention are not particularly limited as long as the object of the present invention can be achieved (e.g., a differentiation-promoting effect is obtained). That is, for example, the amount of the active ingredient used in the method of the present invention may be an effective amount (e.g., an amount that provides a differentiation-promoting effect). Specifically, for example, the concentration of the active ingredient in the medium may be an effective concentration (e.g., a concentration that provides a differentiation-promoting effect). The amount used and the ratio used of each component in the method of the present invention can be appropriately set, for example, according to various conditions such as the type of animal cell, the purpose of culturing the animal cells, and the type of component.

The concentration of the active ingredient in the medium may be, for example, 0.05 nM or more, 0.1 nM or more, 0.2 nM or more, 0.5 nM or more, 1 nM or more, 2 nM or more, 5 nM or more, 10 nM or more, 20 nM or more, 50 nM or more, 100 nM or more, 200 nM or more, 500 nM or more, 1 μM or more, 2 μM or more, 5 μM or more, 10 μM or more, 20 μM or more, 50 μM or more, 100 μM or more, 200 μM or more, 500 μM or more, 1 mM or more, 2 mM or more, 5 mM or more, 10 mM or more, 20 mM or more, 50 mM or more, 100 mM or more, or 200 mM or more, and may be 500 mM or less, 200 mM or less, 100 mM or less, 50 mM or less, 20 mM or less, 10 mM or less, 5 mM or less, 2 mM or less, 1 mM or less, 500 μM or less, 200 μM or less, 100 μM or less, 50 μM or less, 20 μM or less, 10 μM or less, 5 μM or less, 2 μM or less, 1 μM or less, 500 nM or less, 200 nM or less, 100 nM or less, 50 nM or less, 20 nM or less, 10 nM or less, 5 nM or less, 2 nM or less, 1 nM or less, 0.5 nM or less, 0.2 nM or less, or 0.1 nM or less, or any non-contradictory combination thereof. Specifically, the concentration of the active ingredient in the medium may be, for example, 0.05 nM to 0.1 nM, 0.1 nM to 0.2 nM, 0.2 nM to 0.5 nM, 0.5 nM to 1 nM, 1 nM to 2 nM, 2 nM to 5 nM, 5 nM to 10 nM, 10 nM to 20 nM, 20 nM to 50 nM, 50 nM to 100 nM, 100 nM to 200 nM, 200 nM to 500 nM, 500 nM to 1 μM, 1 μM to 2 μM, 2 μM to 5 μM, 5 μM to 10 μM, 10 μM to 20 μM, 20 μM to 50 μM, 50 μM to 100 μM, 100 μM to 200 μM, 200 μM to 500 μM, 500 μM to 1 mM, 1 mM to 2 mM, 2 mM to 5 mM, 5 mM to 10 mM, 10 mM to 20 mM, 20 mM to 50 mM, 50 mM to 100 mM, 100 mM to 200 mM, or 200 mM to 500 mM. Specifically, the concentration of the active ingredient in the medium may be, for example, 0.05 nM to 500 mM, 0.2 nM to 100 mM, or 1 μM to 20 mM. When the medium contains two or more active ingredients, the concentration of these two or more active ingredients in the medium may be set, independently or in total, within the range of the concentration of the active ingredient in the medium exemplified above. When the medium contains two or more active ingredients, “the concentration of the active ingredient in the medium” shall, unless otherwise specified, mean the total concentration of these two or more active ingredients in the medium.

The concentration of an umami substance or umami receptor agonist, such as ATF100, or a kokumi substance or CaSR agonist, such as cinacalcet, in the medium may be, for example, within the range of the concentration of the active ingredient in the medium exemplified above. The concentration of an umami substance or umami receptor agonist, such as ATF100, or a kokumi substance or CaSR agonist, such as cinacalcet, in the medium may be, for example, 0.1 nM or more, 0.2 nM or more, 0.5 nM or more, 1 nM or more, 2 nM or more, 5 nM or more, 10 nM or more, 20 nM or more, 50 nM or more, 100 nM or more, 200 nM or more, 500 nM or more, or 1 μM or more, and may be 2 μM or less, 1 μM or less, 500 nM or less, 200 nM or less, 100 nM or less, 50 nM or less, 20 nM or less, 10 nM or less, 5 nM or less, 2 nM or less, 1 nM or less, 0.5 nM or less, or 0.2 nM or less, or any non-contradictory combination thereof. Specifically, the concentration of an umami substance or umami receptor agonist, such as ATF100, or a kokumi substance or CaSR agonist, such as cinacalcet, in the medium may be, for example, 0.1 nM to 0.2 nM, 0.2 nM to 0.5 nM, 0.5 nM to 1 nM, 1 nM to 2 nM, 2 nM to 5 nM, 5 nM to 10 nM, 10 nM to 20 nM, 20 nM to 50 nM, 50 nM to 100 nM, 100 nM to 200 nM, 200 nM to 500 nM, 500 nM to 1 μM, or 1 μM to 2 μM. Specifically, the concentration of an umami substance or umami receptor agonist, such as ATF100, or a kokumi substance or CaSR agonist, such as cinacalcet, in the medium may be, for example, 0.1 nM to 2 μM, 0.5 nM to 500 nM, or 2 nM to 100 nM.

The concentration of a sweet substance or sweet taste receptor agonist, such as sucralose, in the medium may be, for example, within the range of the concentration of the active ingredient in the medium exemplified above. The concentration of a sweet substance or sweet taste receptor agonist, such as sucralose, in the medium may be, for example, 100 nM or more, 200 nM or more, 500 nM or more, 1 μM or more, 2 μM or more, 5 μM or more, 10 μM or more, 20 μM or more, 50 μM or more, 100 μM or more, 200 μM or more, 500 μM or more, or 1 mM or more, and may be 2 mM or less, 1 mM or less, 500 μM or less, 200 μM or less, 100 μM or less, 50 μM or less, 20 μM or less, 10 μM or less, 5 μM or less, 2 μM or less, 1 μM or less, 500 nM or less, or 200 nM or less, or any non-contradictory combination thereof. Specifically, the concentration of a sweet substance or sweet taste receptor agonist, such as sucralose, in the medium may be, for example, 100 nM to 200 nM, 200 nM to 500 nM, 500 nM to 1 μM, 1 μM to 2 μM, 2 μM to 5 μM, 5 μM to 10 μM, 10 μM to 20 μM, 20 μM to 50 μM, 50 μM to 100 μM, 100 μM to 200 μM, 200 μM to 500 μM, 500 μM to 1 mM, or 1 mM to 2 mM. Specifically, the concentration of a sweet substance or sweet taste receptor agonist, such as sucralose, in the medium may be, for example, 100 nM to 2 mM, 500 nM to 500 μM, or 2 μM to 100 μM.

The concentration of a fatty taste substance or fatty acid receptor agonist, such as TUG-891, a GABA-B receptor agonist, such as SKF97541, a sour substance or sour taste receptor agonist, such as succinic acid, or a succinate receptor agonist, such as succinic acid, in the medium may be, for example, within the range of the concentration of the active ingredient in the medium exemplified above. The concentration of a fatty taste substance or fatty acid receptor agonist, such as TUG-891, a GABA-B receptor agonist, such as SKF97541, a sour substance or sour taste receptor agonist, such as succinic acid, or a succinate receptor agonist, such as succinic acid, in the medium may be, for example, 10 nM or more, 20 nM or more, 50 nM or more, 100 nM or more, 200 nM or more, 500 nM or more, 1 μM or more, 2 μM or more, 5 μM or more, 10 μM or more, 20 μM or more, or 50 μM or more, and may be 100 μM or less, 50 μM or less, 20 μM or less, 10 μM or less, 5 μM or less, 2 μM or less, 1 μM or less, 500 nM or less, 200 nM or less, 100 nM or less, 50 nM or less, or 20 nM or less, or any non-contradictory combination thereof. Specifically, the concentration of a fatty taste substance or fatty acid receptor agonist, such as TUG-891, a GABA-B receptor agonist, such as SKF97541, a sour substance or sour taste receptor agonist, such as succinic acid, or a succinate receptor agonist, such as succinic acid, in the medium may be, for example, 10 nM to 20 nM, 20 nM to 50 nM, 50 nM to 100 nM, 100 nM to 200 nM, 200 nM to 500 nM, 500 nM to 1 μM, 1 μM to 2 μM, 2 μM to 5 μM, 5 μM to 10 μM, 10 μM to 20 μM, 20 μM to 50 μM, or 50 μM to 100 μM. Specifically, the concentration of a fatty taste substance or fatty acid receptor agonist, such as TUG-891, a GABA-B receptor agonist, such as SKF97541, a sour substance or sour taste receptor agonist, such as succinic acid, or a succinate receptor agonist, such as succinic acid, in the medium may be, for example, 10 nM to 100 μM, 50 nM to 20 μM, or 200 nM to 5 μM.

The concentration of a bitter substance or bitter taste receptor agonist (especially a T2R108 agonist), such as diphenidol, in the medium may be, for example, within the range of the concentration of the active ingredient in the medium exemplified above. The concentration of a bitter substance or bitter taste receptor agonist (especially a T2R108 agonist), such as diphenidol, in the medium may be, for example, 10 nM or more, 20 nM or more, 50 nM or more, 100 nM or more, 200 nM or more, 500 nM or more, 1 μM or more, 2 μM or more, 5 μM or more, 10 μM or more, 20 μM or more, 50 μM or more, or 100 μM or more, and may be 200 μM or less, 100 μM or less, 50 μM or less, 20 μM or less, 10 μM or less, 5 μM or less, 2 μM or less, 1 μM or less, 500 nM or less, 200 nM or less, 100 nM or less, 50 nM or less, or 20 nM or less, or any non-contradictory combination thereof. Specifically, the concentration of a bitter substance or bitter taste receptor agonist (especially a T2R108 agonist), such as diphenidol, in the medium may be, for example, 10 nM to 20 nM, 20 nM to 50 nM, 50 nM to 100 nM, 100 nM to 200 nM, 200 nM to 500 nM, 500 nM to 1 μM, 1 μM to 2 μM, 2 μM to 5 μM, 5 μM to 10 μM, 10 μM to 20 μM, 20 μM to 50 μM, 50 μM to 100 μM, or 100 μM to 200 μM. Specifically, the concentration of a bitter substance or bitter taste receptor agonist (especially a T2R108 agonist), such as diphenidol, in the medium may be, for example, 10 nM to 200 μM, 50 nM to 50 μM, or 200 nM to 10 μM.

The concentration of a bitter substance or bitter taste receptor agonist (especially a T2R126 agonist), such as salicin, in the medium may be, for example, within the range of the concentration of the active ingredient in the medium exemplified above. The concentration of a bitter substance or bitter taste receptor agonist (especially a T2R126 agonist), such as salicin, in the medium may be, for example, 5 μM or more, 10 μM or more, 20 μM or more, 50 μM or more, 100 μM or more, 200 μM or more, 500 μM or more, 1 mM or more, 2 mM or more, 5 mM or more, 10 mM or more, 20 mM or more, or 50 mM or more, and may be 100 mM or less, 50 mM or less, 20 mM or less, 10 mM or less, 5 mM or less, 2 mM or less, 1 mM or less, 500 μM or less, 200 μM or less, 100 μM or less, 50 μM or less, 20 μM or less, or 10 μM or less, or any non-contradictory combination thereof. Specifically, the concentration of a bitter substance or bitter taste receptor agonist (especially a T2R126 agonist), such as salicin, in the medium may be, for example, 5 μM to 10 μM, 10 μM to 20 μM, 20 μM to 50 μM, 50 μM to 100 μM, 100 μM to 200 μM, 200 μM to 500 μM, 500 μM to 1 mM, 1 mM to 2 mM, 2 mM to 5 mM, 5 mM to 10 mM, 10 mM to 20 mM, 20 mM to 50 mM, or 50 mM to 100 mM. Specifically, the concentration of a bitter substance or bitter taste receptor agonist (especially a T2R126 agonist), such as salicin, in the medium may be, for example, 5 μM to 100 mM, 20 μM to 20 mM, or 100 μM to 5 mM.

The concentration of a salty substance or salty taste receptor agonist in the medium may be, for example, within the range of the concentration of the active ingredient in the medium exemplified above.

Any of the various components, such as the active ingredient, may be contained in the medium for the entire duration of the culture, or may be contained in the medium for only a part of the culture period. That is, “culturing is carried out in a medium containing a certain component” means that it is sufficient for said component to be contained in the medium for at least a part of the culture period, and it is not required for said component to be contained in the medium for the entire duration of the culture. Any of the various components, such as the active ingredient, may, for example, be contained in the medium at the start of culturing, or may be supplied to the medium after the start of culturing. Also, any of the various components, such as the active ingredient, may, for example, be contained in the medium at the start of culturing and also be further supplied to the medium after the start of culturing (e.g., after consumption of the active ingredient).

Any of the various components, such as the active ingredient, may be contained in the medium at the exemplified concentrations for the entire duration of the culture, for example, or may be contained in the medium at the exemplified concentrations for only a part of the culture period. That is, “culturing is carried out in a medium containing a certain component at a certain concentration,” “a certain component is contained in the medium during culturing at a certain concentration,” or “the concentration of a certain component in the medium during culturing is a certain concentration” means that it is sufficient for the concentration of said component in the medium to be within the range of said concentration for at least a part of the culture period, and it is not required for the concentration of said component in the medium to be within the range of said concentration for the entire duration of the culture. Any of the various components, such as the active ingredient, may, for example, be contained in the medium at the exemplified concentrations at the start of culturing, or may be supplied to the medium after the start of culturing so as to reach the exemplified concentrations. Also, any of the various components, such as the active ingredient, may, for example, be contained in the medium at the exemplified concentrations at the start of culturing and also be further supplied to the medium after the start of culturing (e.g., after consumption of said component) so as to reach the exemplified concentrations.

The length of “a part of the culture period” is not particularly limited as long as the object of the present invention can be achieved (e.g., a differentiation-promoting effect is obtained). The length of “a part of the culture period” can be appropriately set according to various conditions such as the type of animal cell and the length of the culture period. “A part of the period” may be, for example, a period of 10% or more, 20% or more, 30% or more, 40% or more, 50% or more, 60% or more, 70% or more, 80% or more, 90% or more, 95% or more, 97% or more, or 99% or more of the total culture period. “A part of the period” may also be, for example, a period of 10% or more, 20% or more, 30% or more, 40% or more, 50% or more, 60% or more, 70% or more, 80% or more, 90% or more, 95% or more, 97% or more, or 99% or more of the total culture period after the animal cells have differentiated into myoblasts, myocytes, or myotubes. “A part of the period” may also be, for example, a period of 0.5 days or more, 1 day or more, 2 days or more, 3 days or more, 4 days or more, 5 days or more, 6 days or more, 7 days or more, 8 days or more, 9 days or more, 10 days or more, 12 days or more, or 15 days or more.

The concentration of any of the various components, such as the active ingredient, in the medium may be set to the exemplified concentrations, for example, as an average value over a specific period during culturing. That is, “culturing is carried out in a medium containing a certain component at a certain concentration,” “a certain component is contained in the medium during culturing at a certain concentration,” or “the concentration of a certain component in the medium during culturing is a certain concentration” may mean that the average value of the concentration of said component in the medium over a specific period during culturing is within the range of said concentration. “The average value of the concentration of a certain component in the medium over a specific period during culturing” is not particularly limited as long as it can grasp the fluctuation of the concentration of said component during a specific period of culturing, but may mean, for example, the average value of the concentration of said component in the medium measured every 60 minutes, 30 minutes, 20 minutes, or 10 minutes over a specific period during culturing. Examples of “a specific period during culturing” include the entire culture period and a part of the culture period. “A part of the culture period” is as described above.

Any of the various components, such as the active ingredient, may be supplied to the medium throughout the entire culture period, or may be supplied to the medium only during a part of the culture period. “A part of the culture period” is as described above. Any of the various components, such as the active ingredient, may be supplied to the medium continuously, for example, or may be supplied to the medium intermittently. Any of the various components, such as the active ingredient, may be supplied to the medium daily, for example, or may be supplied to the medium every few days.

The concentration of any of the various components, such as the active ingredient, in the fed-batch or perfusion medium may be, for example, within the range of the concentration of said component in the medium exemplified above. The concentration of any of the various components, such as the active ingredient, in the fed-batch or perfusion medium may be, for example, 1 time or more, 1.1 times or more, 1.3 times or more, 1.5 times or more, 2 times or more, 3 times or more, 5 times or more, 7 times or more, 10 times or more, 15 times or more, or 20 times or more the concentration of said component in the medium exemplified above, and may be 100 times or less, 70 times or less, 50 times or less, 30 times or less, 20 times or less, 15 times or less, 10 times or less, 7 times or less, 5 times or less, 3 times or less, or 2 times or less the concentration, or any non-contradictory combination thereof. Specifically, the concentration of any of the various components, such as the active ingredient, in the fed-batch or perfusion medium may be, for example, 1 to 2 times, 1.1 to 2 times, 1.3 to 2 times, 1.5 to 2 times, 2 to 3 times, 3 to 5 times, 5 to 7 times, 7 to 10 times, 10 to 15 times, 15 to 20 times, 20 to 30 times, 20 to 50 times, 20 to 70 times, or 20 to 100 times the concentration of said component in the medium exemplified above. Specifically, the concentration of any of the various components, such as the active ingredient, in the fed-batch or perfusion medium may be, for example, 1 to 100 times, 2 to 50 times, or 5 to 20 times the concentration of said component in the medium exemplified above.

The concentration of any of the various components, such as the active ingredient, can be measured, for example, by a known method used for the detection or identification of compounds. Such methods include HPLC, UPLC, LC/MS, GC/MS, and NMR.

Animal cells can be cultured as described above. In one embodiment, a product (e.g., cultured meat) may be obtained by culturing animal cells as described above.

### <4> Use of the Active Ingredient

The present invention also discloses the use of the active ingredient for the applications exemplified above. That is, the present invention discloses, for example, the use of the active ingredient for culturing animal cells, the use of the active ingredient for promoting the differentiation of animal cells, and the use of the active ingredient in the production of the composition of the present invention, such as a composition for animal cell culture.

The present invention also discloses an active ingredient for use in the applications exemplified above. That is, the present invention discloses, for example, an active ingredient for use in culturing animal cells, an active ingredient for use in promoting the differentiation of animal cells, and an active ingredient for use in the production of a composition for animal cell culture.

### EXAMPLES

Hereinafter, the present invention will be described more specifically with reference to non-limiting examples.

### (1) Evaluation of the Activity of Taste Substances on Muscle Differentiation Using Bovine and Chicken Muscle Stem Cells

### (1-1) Preparation of Bovine and Chicken Muscle Stem Cells

Muscle tissue was collected from edible bovine shank meat, and CD56-positive cells were isolated using a Magnetic-Activated Cell Sorting (MACS) system to obtain bovine muscle stem cells. Separately, muscle stem cells were isolated from edible chicken breast meat.

### (1-2) Evaluation of the Activity of Taste Substances Using Bovine Muscle Stem Cells

The bovine muscle stem cells obtained in (1-1) were suspended in a serum medium. As the serum medium, a medium was used in which Advanced DMEM (Thermo Fisher Scientific, Cat No. 12491015) was supplemented with 20% FBS (Thermo Fisher Scientific, Cat No. 10270106), 10% horse serum (Thermo Fisher Scientific, Cat No. 16050-130), and 1% GlutaMAX™ Supplement (Thermo Fisher Scientific, Cat No. 35050061). The cell suspension was seeded into a 96-well plate coated with 1% Matrigel (Corning, Cat No. 356234) at a cell density of 8,000 cells/well, and the medium was changed to a serum differentiation medium or a serum-free differentiation medium on the next day. As the serum differentiation medium, a medium was used in which DMEM/F12 (Thermo Fisher Scientific, Cat No. 08460-95) was supplemented with 2% FBS (Thermo Fisher Scientific, Cat No. 26140-079). As the serum-free differentiation medium, a medium was used in which DMEM/F12 was supplemented with 1% NEAA (Thermo Fisher Scientific, Cat No. 11140050), 1 mM Glutamine (Ajinomoto Healthy Supply), 73 mg/mL 0.2% selenium yeast (Medience), 2.5 g/L pea protein (Roquette, Product name: NUTRALYS S85F), 30 ng/ml IGF-1 (Laurus Bio, Cat No. Rc LR3-IGF-1), and 10 μg/mL Transferrin (Laurus Bio, Cat No. RTRF-P). Furthermore, to evaluate the taste substances, ATF100 (N—(R)-1-(2-(pyridin-2-yl)ethylcarbamoyl)-2-(benzyloxy)ethyl-4-methoxy-3-methylbenzamide; AJINOMOTO), Sucralose (SIGMA, Cat No. 69293-100G), Cinacalcet (SIGMA, Cat No. SML2012), TUG-891 (SIGMA, Cat No. SML1914), Diphenidol (MedchemExpress, Cat No. HY-A0082), Salicin (SIGMA, Cat No. S0625-25G), SKF97541 (Tocris Bioscience, Cat No. 556-88751), or Sodium succinate (SIGMA, Cat No. 224731-100G) was added to the serum-free differentiation medium. Table 1 summarizes the group numbers and additives.

Then, on the second day, the expression of myosin heavy chain (MyHC) was measured by immunocytochemistry. The cells were fixed with 4% paraformaldehyde (Nacalai Tesque, Cat No. 09154-85) for 15 minutes. After washing twice with Dulbecco's phosphate-buffered saline (without Ca, Mg) (Nacalai Tesque, Cat No. 14249-24), the cells were immersed in Dulbecco's phosphate-buffered saline (without Ca, Mg) containing 0.1% Triton X-100 (Sigma, Cat No. T9284) for 10 minutes. Then, the solution was replaced with a blocking buffer and left to stand for 1 hour. As the blocking buffer, a solution was used in which Dulbecco's phosphate-buffered saline (without Ca, Mg) was supplemented with 5% horse serum and 0.1% Triton X-100. Then, the solution was replaced with a blocking buffer containing 0.2% Anti-Myosin Heavy Chain Antibody, clone A4.1025 (Merck, Cat No. 05-716-I-25UL) and left to stand overnight at 4° C. On the next day, after washing three times with Dulbecco's phosphate-buffered saline (without Ca, Mg), the solution was replaced with a blocking buffer containing 0.1% Goat anti-Mouse IgG2a Antibody, Alexa Fluor 555 (Thermo Fisher Scientific, Cat No. A21137) and 0.1% Cellstain Hoechst 33342 solution (Dojindo Laboratories, Cat No. H342) and left to stand at room temperature for 1 hour. Then, after washing three times with Dulbecco's phosphate-buffered saline (without Ca, Mg), the solution was replaced with Dulbecco's phosphate-buffered saline (without Ca, Mg). MyHC-positive cells were detected with a fluorescence microscope ECLIPSE Ti2-E (Nikon), and the Fusion Index (=Number of MyHC-positive nuclei/Total number of nuclei×100(%)) was calculated.

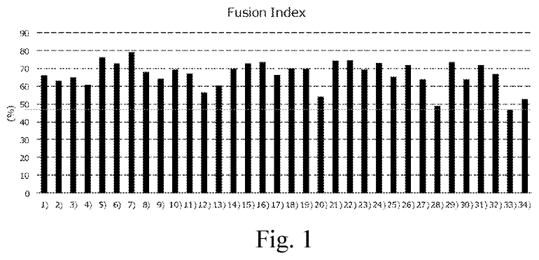

The results are shown in FIG. 1. All of the taste substances promoted the muscle differentiation of bovine cells. It is known that intracellular calcium ions are involved in muscle differentiation (Sansrity Sinha et al., Ca2+ as a coordinator of skeletal muscle differentiation, fusion and contraction. FEBS J. 2022 November; 289 (21): 6531-6542.). It is also known that taste substances cause an increase in intracellular calcium ion concentration in taste cells via binding with taste receptors on the taste cells (Rais Ahmad and Julie E Dalziel, G Protein-Coupled Receptors in Taste Physiology and Pharmacology. Front Pharmacol. 2020 Nov. 30; 11:587664.). That is, in this example, it is considered that the intracellular calcium ion concentration in the muscle stem cells was increased via the binding between the taste substance and the taste receptor on the muscle stem cells, thereby promoting the muscle differentiation of the muscle stem cells. Therefore, it is considered that the use of a taste substance can promote differentiation, such as muscle differentiation, in cells that express the corresponding taste receptor, for example.

TABLE 1

| Group No. | Medium | Additive |
|---|---|---|
| 1) | Serum-free differentiation medium | 0.1 μM ATF100 |
| 2) | Serum-free differentiation medium | 0.033 μM ATF100 |
| 3) | Serum-free differentiation medium | 0.011 μM ATF100 |
| 4) | Serum-free differentiation medium | 0.0037 μM ATF100 |
| 5) | Serum-free differentiation medium | 0.1 mM Sucralose |
| 6) | Serum-free differentiation medium | 0.033 mM Sucralose |
| 7) | Serum-free differentiation medium | 0.011 mM Sucralose |
| 8) | Serum-free differentiation medium | 0.0037 mM Sucralose |
| 9) | Serum-free differentiation medium | 0.1 μM Cinacalcet |
| 10) | Serum-free differentiation medium | 0.033 μM Cinacalcet |
| 11) | Serum-free differentiation medium | 0.011 μM Cinacalcet |
| 12) | Serum-free differentiation medium | 0.0037 μM Cinacalcet |
| 13) | Serum-free differentiation medium | 5 μM TUG-891 |
| 14) | Serum-free differentiation medium | 1.67 μM TUG-891 |
| 15) | Serum-free differentiation medium | 0.56 μM TUG-891 |
| 16) | Serum-free differentiation medium | 0.19 μM TUG-891 |
| 17) | Serum-free differentiation medium | 10 μM Diphenidol |
| 18) | Serum-free differentiation medium | 3.3 μM Diphenidol |
| 19) | Serum-free differentiation medium | 1.1 μM Diphenidol |
| 20) | Serum-free differentiation medium | 0.37 μM Diphenidol |
| 21) | Serum-free differentiation medium | 2.5 mM Salicin |
| 22) | Serum-free differentiation medium | 0.83 mM Salicin |
| 23) | Serum-free differentiation medium | 0.28 mM Salicin |
| 24) | Serum-free differentiation medium | 0.093 mM Salicin |
| 25) | Serum-free differentiation medium | 5 μM SKF97541 |
| 26) | Serum-free differentiation medium | 1.67 μM SKF97541 |
| 27) | Serum-free differentiation medium | 0.56 μM SKF97541 |
| 28) | Serum-free differentiation medium | 0.19 μM SKF97541 |
| 29) | Serum-free differentiation medium | 5 μM Sodium succinic acid |
| 30) | Serum-free differentiation medium | 1.67 μM Sodium succinic acid |
| 31) | Serum-free differentiation medium | 0.56 μM Sodium succinic acid |
| 32) | Serum-free differentiation medium | 0.19 μM Sodium succinic acid |
| 33) | Serum-free differentiation medium | No additive |
| 34) | Serum-containing differentiation medium | No additive |

### (1-3) Evaluation of the Activity of Taste Substances Using Chicken Muscle Stem Cells

The chicken muscle stem cells obtained in (1-1) were suspended in a serum medium. As the serum medium, a medium was used in which DMEM (Nacalai Tesque, Cat No. 08456-65) was supplemented with 10% chicken serum (Thermo Fisher Scientific, Cat No. 16110-082) and 5% horse serum. The cell suspension was seeded into a 96-well plate coated with 10 μg/cm² gelatin (Sigma, Cat No. G1393-20ML) at a cell density of 60,000 cells/well, and the medium was changed to a serum differentiation medium or a serum-free differentiation medium on the next day. As the serum differentiation medium, a medium was used in which DMEM/F12 was supplemented with 2% FBS. As the serum-free differentiation medium, a medium was used in which DMEM/F12 was supplemented with 1% NEAA (Thermo Fisher Scientific, Cat No. 11140050), 1 mM Glutamine (Ajinomoto Healthy Supply), 73 mg/ml 0.2% selenium yeast (Medience), 30 ng/ml IGF-1 (Laurus Bio, Cat No. Rc LR3-IGF-1), and 10 μg/mL Transferrin (Laurus Bio, Cat No. RTRF-P). Furthermore, to evaluate the taste substances, ATF100, Sucralose, Cinacalcet, Cinacalcet, TUG-891, Diphenidol, Salicin, SKF97541, or Sodium succinate was added to the serum-free differentiation medium. Table 1 summarizes the group numbers and additives.

Then, on the second day, the expression of myosin heavy chain (MyHC) was measured by the same method as in (1-2).

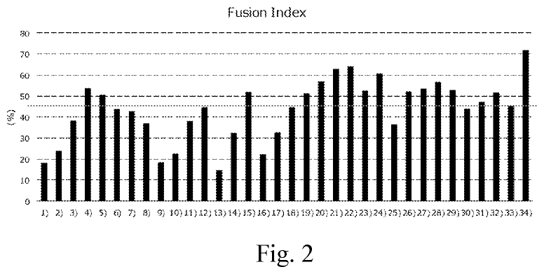

The results are shown in FIG. 2. Only Salicin promoted the muscle differentiation of chicken cells.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

### INDUSTRIAL APPLICABILITY

According to the present invention, the differentiation of animal cells can be promoted.

1. A medium or a medium supplement comprising a taste substance.

2. The medium or the medium supplement according to claim 1, wherein the taste substance is at least one component selected from the group consisting of a sweet substance, a salty substance, a sour substance, a bitter substance, an umami substance, a fatty taste substance, a kokumi substance, a succinate receptor agonist, and a GABA-B receptor agonist.

3. The medium or the medium supplement according to claim 2, wherein
the sweet substance is at least one component selected from the group consisting of a sugar, a sugar alcohol, a high-intensity sweetener, and an amino acid,
the salty substance is at least one inorganic salt,
the sour substance is at least one component selected from the group consisting of an organic acid, an inorganic acid, and an amino acid,
the bitter substance is at least one component selected from the group consisting of an amino acid, a polyphenol, an alkaloid, a terpenoid, an inorganic salt, diphenidol, and salicin,
the umami substance is at least one component selected from the group consisting of an amino acid, a nucleic acid, an organic acid, an amide compound, and a high-potency umami substance,
the fatty taste substance is at least one component selected from the group consisting of a fatty acid and a fatty acid receptor agonist,
the kokumi substance is at least one component selected from the group consisting of a γ-glutamyl peptide and a calcium-sensing receptor agonist,
the succinate receptor agonist is succinic acid, and
the GABA-B receptor agonist is GABA and/or SKF97541.

4. The medium or the medium supplement according to claim 1, wherein the taste substance is at least one component selected from the group consisting of a sweet taste receptor agonist, a salty taste receptor agonist, a sour taste receptor agonist, a bitter taste receptor agonist, an umami taste receptor agonist, a fatty acid receptor agonist, a calcium-sensing receptor agonist, a succinate receptor agonist, and a GABA-B receptor agonist.

5. The medium or the medium supplement according to claim 1, wherein the taste substance is at least one component selected from the group consisting of ATF100, sucralose, cinacalcet, TUG-891, diphenidol, salicin, SKF97541, and succinic acid.

6. The medium or the medium supplement according to claim 1, wherein the taste substance is a taste substance other than a sugar, an amino acid, and calcium.

7. The medium or the medium supplement according to claim 1, wherein the medium is an animal cell culture medium.

8. The medium or the medium supplement according to claim 1, wherein the composition is substantially free of animal-derived albumin.

9. The medium or the medium supplement according to claim 1, further comprising one or more components selected from the group consisting of carbon sources, amino acid sources, peptides, proteins, vitamins, fatty acids, lipids, organic acids, nucleic acids, amines, antioxidants, inorganic components, pH buffering agents, growth factors, cytokines, hormones, cell adhesion factors, extracellular matrix components, serum, lecithin, pea-derived materials, yeast extract, antibiotics, and gene expression inducers.

10. A method for producing cultured meat, comprising the step of:
culturing animal cells in the presence of a taste substance to differentiate the animal cells.

11. A method for promoting differentiation of animal cells, comprising the step of:
culturing animal cells in the presence of a taste substance to differentiate the animal cells.

12. The method according to claim 10, wherein the taste substance is at least one component selected from the group consisting of a sweet substance, a salty substance, a sour substance, a bitter substance, an umami substance, a fatty taste substance, a kokumi substance, a succinate receptor agonist, and a GABA-B receptor agonist.

13. The method according to claim 12, wherein
the sweet substance is at least one component selected from the group consisting of a sugar, a sugar alcohol, a high-intensity sweetener, and an amino acid,
the salty substance is at least one inorganic salt,
the sour substance is at least one component selected from the group consisting of an organic acid, an inorganic acid, and an amino acid,
the bitter substance is at least one component selected from the group consisting of an amino acid, a polyphenol, an alkaloid, a terpenoid, an inorganic salt, diphenidol, and salicin,
the umami substance is at least one component selected from the group consisting of an amino acid, a nucleic acid, an organic acid, an amide compound, and a high-potency umami substance,
the fatty taste substance is at least one component selected from the group consisting of a fatty acid and a fatty acid receptor agonist,
the kokumi substance is at least one component selected from the group consisting of a γ-glutamyl peptide and a calcium-sensing receptor agonist,
the succinate receptor agonist is succinic acid, and
the GABA-B receptor agonist is GABA and/or SKF97541.

14. The method according to claim 10, wherein the taste substance is at least one component selected from the group consisting of a sweet taste receptor agonist, a salty taste receptor agonist, a sour taste receptor agonist, a bitter taste receptor agonist, an umami taste receptor agonist, a fatty acid receptor agonist, a calcium-sensing receptor agonist, a succinate receptor agonist, and a GABA-B receptor agonist.

15. The method according to claim 10, wherein the taste substance is at least one component selected from the group consisting of ATF100, sucralose, cinacalcet, TUG-891, diphenidol, salicin, SKF97541, and succinic acid.

16. The method according to claim 10, wherein the taste substance is a taste substance other than a sugar, an amino acid, and calcium.

17. The method according to claim 10, wherein the concentration of the taste substance in the medium during culturing is from 0.05 nM to 500 mM.

18. The method according to claim 10, wherein the differentiation is muscle differentiation.

19. The method according to claim 10, wherein the cell is a satellite cell or a myoblast.

20. The method according to claim 10, wherein the culturing is performed in a medium substantially free of animal-derived albumin.
